(19) United States
(12) Patent Application Publication
(10) Pub. No.: US 2016/0107159 A1
(43) Pub. Date: Apr. 21, 2016

(54) MICROFLUIDIC DEVICE AND METHOD FOR CONTROLLING FLUID FLOW THEREINTO

(71) Applicant: STAR ARRAY PTE LTD, Singapore (SG)

(72) Inventor: Haiqing GONG, Singapore (SG)

(73) Assignee: STAR ARRAY PTE LTD, Singapore (SG)

(21) Appl. No.: 14/894,296

(22) PCT Filed: May 27, 2013
(86) PCT No.: PCT/SG2013/000213

§ 371 Date: Nov. 25, 2015

(51) Int. Cl. B01L 3/00; C12Q 1/68

(52) U.S. Cl. CPC B01L 3/50273; C12Q 1/6806; B01L 3/567; B01L 2400/049; B01L 2300/14; B01L 2400/06; B01L 2300/04; B01L 2200/0642

(57) ABSTRACT

A microfluidic device (100), which has applications in disease detection and analysis, is disclosed. The device comprises a member (102) with a base having at least one well (110), the at least one well in fluid communication with an adjacent space (112), said space being in fluid communication with at least one channel (114, 118); and a vacuum generating device (108) coupled to the at least one channel. The vacuum generating device is configured to generate first and second absolute pressures that are each lower than atmospheric pressure at a first and a second regions of the microfluidic device respectively, wherein the first absolute pressure is higher than the second absolute pressure, thus generating a differential pressure between the first and second region of the microfluidic device to control a speed of a fluid flowing through the space of the device, for filling the at least one well progressively and/or facilitating retention of any material disposed in the at least one well. The controlled fluid flow also prevents cross contamination of specific biological and/or chemical substances pre-loaded in a plurality of such wells. A related thermocycler and methods are also disclosed.

### FIELD OF THE INVENTION

The present invention relates to a microfluidic device and method for controlling fluid flow thereinto. It also relates to a thermocycler which incorporates the microfluidic device.

### BACKGROUND OF THE INVENTION

Well plates (also known as microtiter plates) containing arrays of wells have found wide applications in biology or chemistry, in which a variety of tests involving chemical and biological samples may be performed using the well plates. For example, different pairs of polymerase chain reaction (PCR) primers can be preloaded into different wells of a well plate for simultaneous amplification of target nucleic acid molecules in a given sample. Alternatively, the well arrays can be used for any other type of assays, for example cell or antibody assays.

In line with the recent development of high throughput assays, the number of wells configured in such well plates has increased from the prior of less than a hundred to typically a few thousand or more, which accordingly lead to smaller well sizes and higher density well arrays.

Conventionally, manual or robotic pipetting is used to load the fluid sample into the well arrays. However, as the density of the well arrays increases, it becomes more time consuming to complete loading all the well arrays, which typically may include a few hundreds or thousands of wells. Moreover, the greater the density of the well arrays in a well plate, the smaller the size of each well which in turn present difficulties for performing pipetting due to demanding technical requirements such as aligning the tips of pipettes with the smaller-sized wells, and generating small liquid droplets, in a cost-effective manner, for loading into the smaller-sized wells.

Another issue is that conventional wells are normally configured as dead-end wells, which may trap air at corner(s) of the bottom of the wells when the wells shrink in size, since a droplet of sample fluid dispensed into a well may cover the opening of the associated well or a portion of the space near the bottom of the well, thus trapping an air pocket within the well. It will be apparent that the trapped air pocket may negatively affect an assay. For example, under a heating step which is required for nucleic acid amplification like polymerase chain reaction (PCR), trapped air pockets can cause the sample fluid to evaporate into the vicinity of the air pockets, and consequently cause the air pockets to expand and push the liquid sample out of the well.

Besides the manner of trapping air pockets as outlined above, loading fluid sample into the wells can also further trap air pockets therewithin during the process. Specifically, liquid sample loading devices, that will give rise to the immediate aforementioned issue, typically have wells that are connected by a common channel of a headspace, and the fluid sample then enters the wells through the common channel. Air may subsequently be trapped in the wells due to movement of the fluid sample over the top of the wells (which undesirably prevents access to the wells through the openings), or to the hydrophobic nature of the surface of the wells that prevent the fluid sample from wetting entire surface of the wells.

To facilitate the fluid sample to flow into the wells, vacuum can be applied to remove air from the wells prior to the sample loading into the well. However, vacuuming the wells and the space or a channel connecting the wells can generate a large pressure differential between the vacuumed wells and the chamber storing the sample fluid which is under atmospheric pressure. During sample loading, such a large pressure differential can cause a sample to flow into the well-connecting space/channel and the associated wells at high speed. Such high speed flow can typically flush the pre-loaded materials inside the well out of the wells, causing failure of assays to be carried out in the wells.

Retaining materials pre-loaded in the wells is important. For many biological and chemical applications using the well arrays, the wells may be preloaded with specific (e.g. different PCR primers or proteins or antibodies in different wells) or non-specific materials (e.g. the same PCR primers in all the wells, Taq polymerase enzyme, cells, proteins or chemical reaction components), and some of these materials are typically freeze-dried before the fluid sample is introduced to fill the wells. It will be apparent that it is important to retain those materials inside their intended wells during a process of introducing of the fluid sample. When a large vacuum is applied to the wells and well-connecting space to remove air in the wells to facilitate sample to flow into the wells, the large pressure differential causes the fluid sample to flow at high speed into the wells and flush out (some of) the materials from the wells, causing loss of those materials from the wells or undesirably moving those materials from one well to another thereby resulting in cross-contamination of some of the materials specific to certain wells.

To retain the preloaded materials in their specific wells, it is important for fluid sample intended for those wells to fill and thereafter be retained within, since loss of a portion of the fluid sample as filled in an associated well may flush the preloaded materials out into neighbouring wells or out of a chip (in which the wells are formed thereon) into connecting channels.

The higher a vacuum level is desired (further below atmospheric pressure), the higher the sample loading speed that may impact into the wells and flush out the pre-loaded material. For example, for a desirable vacuum level of 10 torr in an array of wells of 0.5 mm×0.5 mm×0.5 mm in dimensions, the sample (water) flow speed in a gap space of 0.5 mm high that connects all the wells can reach 750 mm per second. Such high speed caused by the desirable vacuum level is undesirable to retaining the pre-loaded material in the wells.

Further, another problem encountered with conventional devices is that air bubbles may appear in many liquid filled fluidic or microfluidic flow path such as flow channels, chambers, and liquid loading ports. These air bubbles can be dragged into the flow path by fluid flowing at the liquid loading ports, or the air bubbles can be trapped by liquid flowing over the flow path surface due to sharp corners, dents, micro-cavities, hydrophobic patches over surfaces of the flow path. Presence of these air bubbles in the flow path may cause adverse effect to a device utilising the flow path. For example, movement of air bubbles in the flow path may disturb the flow field which may be important to maintain a specific particles/cells distribution in the flow field inside the flow path. In a channel to separate biological cells based on hydrodynamic forces such as forces due to Dean flow in a spiral channel, presence of air bubbles in the flow path can disturb the cell location and push undesirable cells into a cell collection outlet. Another adverse effect of the air bubbles in the flow path is the growth in size of the air bubbles upon heating, in which the bubble-water interface encourages water evaporation upon heating and causes the growth in size of the air bubbles.

It is therefore desirable to address some of the problems identified and/or to provide a choice that is useful in the art.

### SUMMARY OF THE INVENTION

According to a first aspect of the invention, there is provided a microfluidic device comprising a member with a base having at least one well, the at least one well in fluid communication with an adjacent space, said space being in fluid communication with at least one channel; and a vacuum generating device coupled to the at least one channel. The vacuum generating device is configured to generate first and second absolute pressures that are each lower than atmospheric pressure at a first and a second region of the microfluidic device respectively, wherein the first absolute pressure is higher than the second absolute pressure, thus generating a differential pressure between the first and second regions of the microfluidic device to control a speed of a fluid flowing through the space of the device, for filling the at least one well progressively and/or facilitating retention of any material disposed in the at least one well.

For example, the vacuum generating device may comprise at least two vacuum generators in cooperative arrangement to generate the differential pressure. Specifically, the at least one channel may include at least first and second channels, a first vacuum generator may be coupled to the at least first channel being an inlet channel for the fluid to flow into the space adjacent the at least one well and a second vacuum generator may be coupled to the at least second channel being an outlet channel for the fluid to flow out of the space adjacent the at least one well. Further, the first vacuum generator may be configured to generate a first absolute pressure in the vicinity of the inlet channel and the second vacuum generator may be configured to generate a second absolute pressure in the vicinity of the outlet channel to control the speed of fluid flow into the space adjacent the at least one well.

In particular, at least one of the two vacuum generators may include a pressure regulator configured to enable independent adjustment of pressure in the vicinity of the inlet channel or the outlet channel. The inlet channel may be connected to a container comprising a reservoir of fluid, while the outlet channel may lead to a container for collecting the fluid. Additionally, the device may further comprise at least one control valve disposed adjacent to the at least one channel to control fluid access into the space. Also, the device may further comprise at least a first control valve, disposed adjacent to the inlet channel, adjustable to allow fluid access to the space and at least a second control valve, disposed adjacent to the outlet channel, adjustable to allow the fluid to flow out of the space.

Accordingly, the differential pressure causes the fluid to flow through the space from the inlet channel to the outlet channel. In particular, the at least one well may be in fluid communication with the space by being connected to the space via at least one channel. The device may further comprise a cover for the member and top and bottom rigid members removably attached to respectively the cover and member of the device to prevent warping under influence of the differential pressure during operation. Additionally, the device may further comprise a substantially airtight chamber to enclose the container therewithin, wherein the container is adapted to be reversibly deformable as the pressure in the chamber is altered. Particularly, the first and second absolute pressures may be vacuum pressures. The device may also further comprise a cover for the member adapted to be moved to reduce a size of the space. In particular, the device may be adapted for thermocycling in a thermocycler. Also, the member may be a microtiter plate. The device may also be adapted to enable fluorescent detection using visible or ultraviolet light to be performed on the at least one well.

According to a second aspect of the invention, there is provided a thermocycler comprising the microfluidic device according to the first aspect of the invention. According to a third aspect of the invention, there is provided a method of controlling fluid flow within a microfluidic device comprising a member with a base having at least one well, the at least one well in fluid communication with an adjacent space, said space being in fluid communication with at least one channel; and a vacuum generating device coupled to the at least one channel. The method comprises generating first and second absolute pressures that are each lower than atmospheric pressure at a first and a second region of the microfluidic device respectively, wherein the first absolute pressure is higher relative to the second absolute pressure thus generating a differential pressure between the first and second regions of the microfluidic device to control a speed of a fluid flowing through the space of the device, for filling the at least one well progressively and/or facilitating retention of any materials that are disposed in the at least one well.

For example, the method may further comprise using the vacuum generating device which comprises at least two vacuum generators in cooperative arrangement to generate the differential pressure. Specifically, the method may comprise using at least a first vacuum generator coupled to at least a first channel being an inlet channel for the fluid to flow into the space adjacent to the at least one well to generate the first absolute-pressure in the vicinity of the inlet channel and at least a second vacuum generator coupled to at least a second channel being an outlet channel for the fluid to flow out of the space adjacent the at least one well to generate the second different absolute pressure in the vicinity of the outlet channel to control the speed of fluid flow into the space adjacent to the at least one well, wherein the at least one channel includes the at least first and second channels. In particular, at least one of the vacuum generators may include a pressure regulator to independently adjust the first absolute pressure or second absolute pressure.

In particular, the method may further comprise controlling fluid access into the space via at least one control valve disposed adjacent to the at least one channel. Also, the method may comprise allowing fluid access into the space by using at least a first control valve disposed adjacent to the inlet channel and allowing the fluid to flow out of the space by using at least a second control valve disposed adjacent to the outlet channel.

Further, the method may comprise introducing a sealant to substantially replace the fluid in the space subsequent to substantially filling the at least one well with the fluid, and filling the space with the sealant to seal the at least one well substantially filled with the fluid, wherein the sealant is introduced using the generated differential pressure or compressed air configured to be of a substantially high pressure for further pushing the fluid filled in the at least one well into any unoccupied space in the at least one well as the sealant fills the space. Alternatively, the method may comprise substantially removing the fluid from the space subsequent to substantially filling the at least one well with the fluid, and introducing a sealant into the space to seal the at least one well substantially filled with the fluid, wherein the sealant is introduced using the generated differential pressure or compressed air configured to be of a substantially high pressure for further pushing the fluid filled in the at least one well into any unoccupied space in the at least one well as the sealant fills the space. Moreover, the method may comprise introducing the sealant from a common container containing both the fluid and the sealant or, introducing the fluid from a first container and introducing the sealant from a separate second container containing the sealant only. The method may further comprise using the differential pressure, to direct fluid flow through the space from the inlet channel to the outlet channel. The method may further comprise substantially removing the fluid from the space subsequent to filling the at least one well with the fluid and moving a cover reduce the space and/or to seal the at least one well substantially filled with the fluid.

According to a fourth aspect of the invention, there is provided a method of controlling fluid flow within a microfluidic device comprising a member with a base having at least one well, the at least one well opening into fluid communication with an adjacent space, said space being in fluid communication with inlet and outlet channels; a fluid dispensing device coupled to the inlet channel; and a vacuum generating device coupled to the outlet channel. The method comprises using the vacuum generating device to generate an absolute pressure lower than atmospheric pressure in the vicinity of the outlet channel; and operating the fluid dispensing device to provide an absolute pressure in the vicinity of the inlet channel which is lower than atmospheric pressure but higher than the absolute pressure in the vicinity of the outlet channel, thus generating a differential pressure to control a speed of flow of the fluid into the space for filling the at least one well progressively and/or facilitating retention of any material disposed in the at least one well.

According to a fifth aspect of the invention, there is provided a microfluidic device comprising a member with a base, and a space which is in fluid communication with the base and at least one channel; and a vacuum generating device coupled to the at least one channel. The vacuum generating device is configured to generate first and second absolute pressures at a first and a second region of the space of the device respectively that are each lower than atmospheric pressure, wherein the first absolute pressure is higher than the second absolute pressure thus generating a differential pressure to control a speed of a fluid flowing through the space of the device.

The fluid may include particles of different respective sizes. Further, the at least one channel may preferably include at least one inlet channel, and the space is arranged as a conduit in fluid communication with the at least one inlet channel which is in fluid communication with a reservoir of the fluid, and the vacuum generating device may further be configured to generate the first absolute pressure at a region of the at least one inlet channel. In addition, the at least one channel may also include at least two outlet channels, and the space is arranged as a conduit in fluid communication with the at least two outlet channels, wherein the microfluidic device is configured to direct the particles of respective sizes to each of the corresponding said outlet channels. For example, particles of different sizes may thus be separated.

According to a sixth aspect of the invention, there is provided a method of controlling fluid flow within a microfluidic device comprising a member with a base, and a space which is in fluid communication with the base and at least one channel; and a vacuum generating device coupled to the at least one channel. The method comprises using the vacuum generating device to generate, first and second absolute pressures at a first and a second region of the space respectively, wherein the first and second absolute pressures are each lower than atmospheric pressure, and the first absolute pressure is higher than the second absolute pressure, thus generating a differential pressure to control a speed of a fluid flowing through the space of the device.

The channel may be of any desired shape. For example, the channel may be substantially straight, u-shaped, s-shaped, zig-zagged, serpentine shaped or spiral shaped.

Preferably, the fluid and the any materials disposed in the at least one well may include constituents that enable biological assays being one of nucleic acid amplification, cell assay and assays involving a plurality of biological particles and chemical agents.

Also, the fluid may include nucleic acid molecules and/or biological cells. On the other hand, the any materials disposed in the at least one well may include primers and/or probes for nucleic acid amplification, or same or different primers and/or probes.

According to a seventh aspect of the invention, there is provided a microfluidic device comprising a member with a base having a plurality of wells, the plurality of wells in fluid communication with an adjacent space, said space being in fluid communication with at least one channel, and a vacuum generating device coupled to the at least one channel. The vacuum generating device is configured to generate first and second absolute pressures that are each lower than atmospheric pressure at a first and a second region of the microfluidic device respectively, wherein the first absolute pressure is higher than the second absolute pressure, thus generating a differential pressure between the first and second regions of the microfluidic device to control a speed of a fluid flowing through the space of the device, for filling the plurality of wells progressively and/or facilitating retention of any material disposed in the plurality of wells. Also, each of the plurality of wells holds specific preloaded material different from those in another well for facilitating nucleic acid amplifications, such as polymerase chain reaction and other primer extensions, and/or assays related to cells and proteins. The material may include cells, proteins and oligonucleotides.

It would be understood that features relating to one aspect of the invention may also be applicable to the other aspects of the invention.

These and other aspects of the invention will be apparent from and elucidated with reference to the embodiments described hereinafter.

### BRIEF DESCRIPTION OF THE DRAWINGS

Embodiments of the invention are disclosed hereinafter with reference to the accompanying drawings, in which:

FIG. 1a is an isometric view of a microfluidic device, according to an embodiment of the invention;

FIG. 1b is an enlarged isometric view of a microtiter plate and cover of the microfluidic device of FIG. 1a;

FIG. 2 is a cross-sectional side elevation view of the microfluidic device of FIG. 1a;

FIG. 3 depicts a section of well arrays of the microfluidic device of FIG. 1a preloaded with various types of biological/chemical materials, depending on different specific applications for the microfluidic device;

FIGS. 4a to 4d illustrate a method for introducing fluid sample into and thereafter sealing the well arrays of the microfluidic device of FIG. 1a;

FIGS. 5a to 5e illustrate another method for introducing fluid sample into and thereafter sealing the well arrays of the microfluidic device of FIG. 1a, according to a further embodiment;

FIG. 6 is a cross-sectional side elevation view of a microfluidic device, according to yet another embodiment;

FIGS. 7a and 7b depict two possible configurations of well arrays of the microfluidic device of FIG. 1a, according to a next different embodiment;

FIGS. 8a and 8b respectively depict an isometric view and schematic arrangement of a microfluidic device, according to yet a different embodiment;

FIGS. 9a to 9e illustrate a method for introducing fluid sample into and thereafter sealing the well arrays of the microfluidic device of FIG. 8a;

FIGS. 10a to 10c illustrate a method for loading multiple biological/chemical materials into the well arrays of the microfluidic device of FIG. 1a, according to an alternative embodiment;

FIG. 11a illustrates a method for controlling a speed of introducing fluid sample into the well arrays of the microfluidic device of FIG. 1a, according to yet a next embodiment;

FIG. 11b illustrates in greater detail the method of FIG. 11a;

FIG. 12 shows a top plan view of a microtiter plate according to an alternative embodiment of the microfluidic device of FIG. 1a;

FIGS. 13a to 13d illustrate a method for introducing fluid sample into and thereafter sealing the well arrays of a microfluidic device, according to a different alternative embodiment; and

FIGS. 14a and 14b show yet another alternative embodiment of the microfluidic device of FIG. 1a, in which no well arrays are arranged in the microfluidic device.

### DETAILED DESCRIPTION OF THE INVENTION

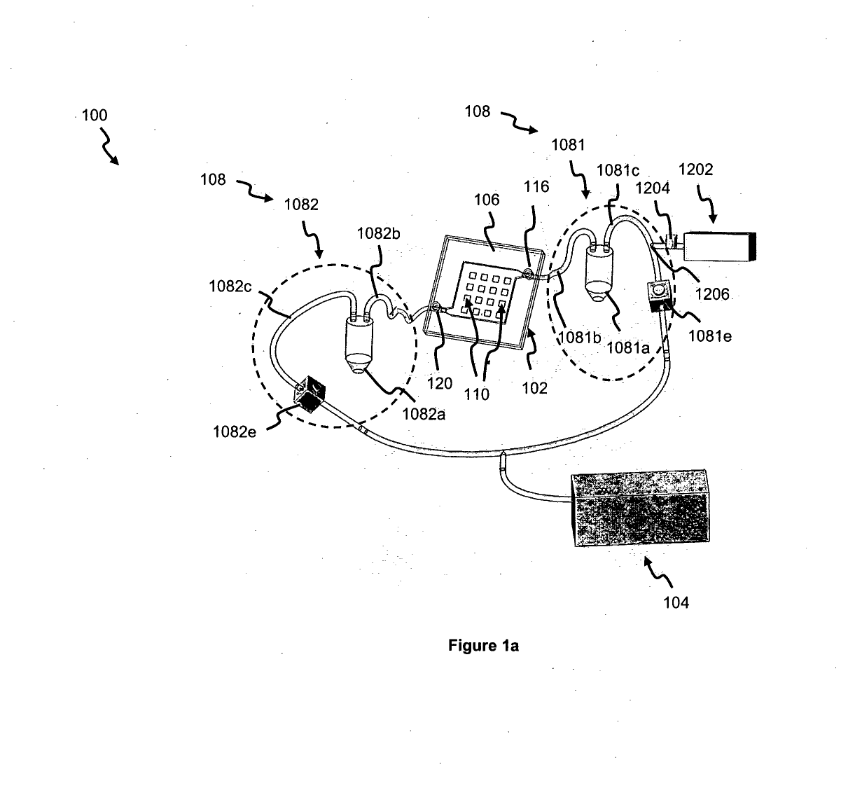

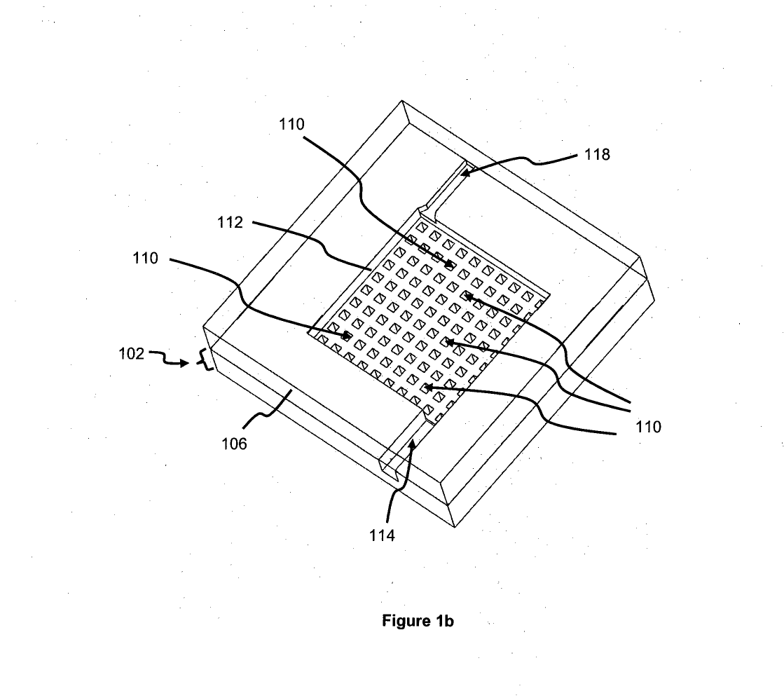

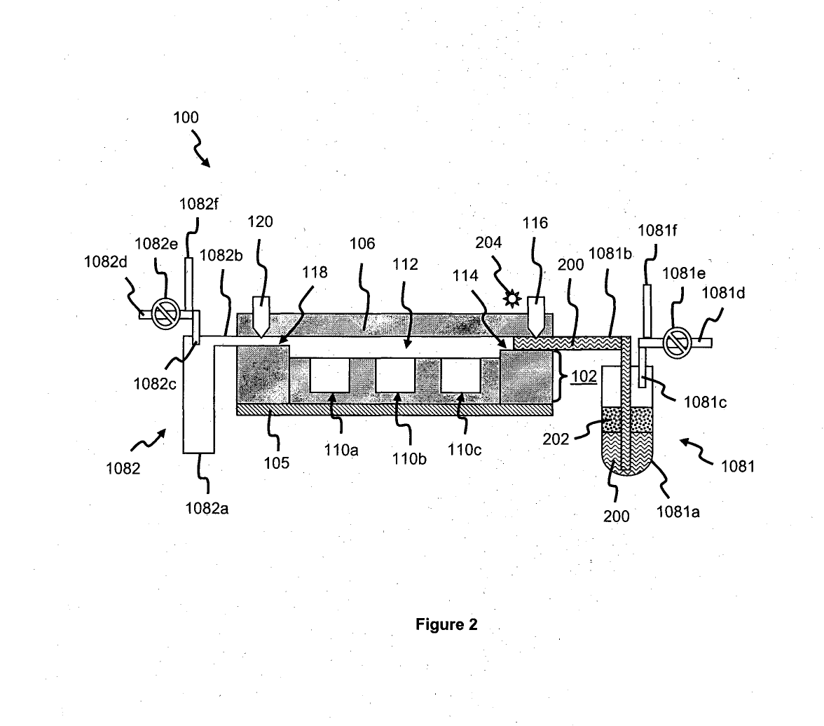

FIGS. 1a and 2 respectively depict an isometric view and a cross-sectional side elevation view of a microfluidic device 100, according to a first embodiment of the invention. The microfluidic device 100 comprises a member 102 (with a base), a cover 106 and a vacuum generating device 108, which includes first and second vacuum generators 1081, 1082. The first and second vacuum generators 1081, 1082 are in turn coupled to a single common vacuum source 104. Specifically, the member 102 is a microtiter plate and will be referred to as such hereinafter. An enlarged isometric view of the microtiter plate 102 and the cover 106 is depicted in FIG. 1b. The microtiter plate 102 can be formed of suitable materials including Polydimethylsiloxane (PDMS), plastics, glass, metal, ceramics and the like. In this embodiment, microtiter plate 102 is realised in the form factor of a chip. The base and cover 106 are of similar shape and size, and more specifically of substantially flat rectangular-like shape. Furthermore, the cover 106 is formed to be substantially transparent. In a typical example, the base is formed with a plurality of wells 110 arranged in an array (hereinafter “array of wells”) in a central portion of the base, wherein each well 110 is of a same size and generally cuboid-shaped, and is adapted to hold biological/chemical materials (in dried, partially dried, or liquid forms), for example PCR primers, cells, viruses, antibodies, proteins, enzymes, molecules, peptides, nucleic acid molecules (e.g. DNA, RNA, mRNA, microRNA, cDNA etc), polynucleotides, oligonucleotides, short fragments of genes, probes etc., reaction constituents, bacteria, protozoa, pathogens, fluorescent chemicals/molecules, crystals, solid particles such as fluorescent particles, fluorescent dye chemicals etc. It will be appreciated that if the biological/chemical materials preloaded into the wells 110 are partially evaporated (or partially dried), it would provide a space in within the wells 110 to allow a fluid sample 200 to flow in when loading thereof. Yet further, the biological/chemical materials can also be in the form of double emulsion droplets or a mixture of water droplets in oil, in which the water droplets contain nucleic acid molecules (e.g. DNA, RNA, mRNA, microRNA, cDNA etc) or constituents, required for nucleic analysis, such as PCR, cells, proteins, antibodies, oligonucleotides, PCR primers and the like. It will however also be understood that certain water droplets need not necessarily contain any nucleic acid molecules or cells, such as when performing nucleic acid amplification techniques (e.g. digital PCR) or single cell analysis. Particularly, it will be appreciated that this is generally useful for loading: (i). the biological/chemical materials (e.g. molecules, cells or drug molecules) or (ii). markers (e.g. PCR primers, cells, antibodies or drug molecules) into the array of wells 110 before the fluid sample 200 is introduced. Each generally cuboid-shaped well 110 is also arranged to be equally spaced apart from immediate neighbouring wells 110, and in this instance, each well 110 has an edge length of approximately between 0.05 μm to 10 mm. It is to be appreciated that for illustration simplicity, only three wells 110a, 110b, 110c in the array of wells 110 are shown in FIG. 2, and unless explicitly specified, reference will be made to the three wells 110a, 110b, 110c (instead of the array of wells 110) for the description hereinafter wherever appropriate, but not to be construed as limiting in any manner.

As will be appreciated, the term “well” 110a, 110b, 110c has a standard meaning known in the art. Specifically, each well 110a, 110b, 110c is a depression for holding the fluid sample 200, and is formed by removing a part of a solid mass (e.g. using chemical/electrochemical etching or sculpting a depression out of a solid mass). The depression can also be formed by moulding or casting a curable liquid to produce a solid mass having the depression (e.g. using a pre-fabricated die to produce a complementary shape). The shape of each well 110a, 110b, 110c is defined by two or more faces. Non-limiting examples of possible shapes for the well 110a, 110b, 110c include cylindrical, conical, pyramid-like, prism-like and truncated variants etc. The shape defining the well 110a, 110b, 110c is arranged with an opening through which fluid can enter/exit the well 110a, 110b, 110c. It is apparent that the opening for the well 110a, 110b, 110c can be rectangular (including square) or circular in shape. Further, where appropriate, the opening is greater in dimension than a lower surface of the well 110a, 110b, 110c. For example, the well 110a, 110b, 110c is shaped as a truncated square pyramid wherein a largest square face provides an opening to the well 110a, 110b, 110c. Embodiments of the present invention (to be subsequently described below) are suited to low, medium and high-density well applications. Low-density well applications typically use less than 50 reaction wells per chip, while medium-density well applications typically use between about 50 to 5000 reaction wells per chip. High-density well applications typically use more than 5000 reaction wells and up to a few million wells per chip. Embodiments of the present invention utilise wells that are each arranged with a volume of about between 0.1 pL to 1 mL. The wells 110a, 110b, 110c are distributed evenly on the microtiter plate 102, in the form of a grid or ordered array to facilitate manufacturing and image recognition during fluorescent detection stages. In particular, the microfluidic device 100 is also adapted to enable fluorescent detection using visible or ultraviolet light to be performed on the wells 110a, 110b, 110c. That is, the visible or ultraviolet light is transmissible into the wells 110a, 110b, 110c for the above mentioned purpose.

It is also to be appreciated that whilst the microtiter plate 102 is designed for both single-use and multi-use applications, the microtiter plate 102 is especially suited for single-use applications. For example, the microtiter plate 102 is constructed of raw materials that are relatively inexpensive, and are substantially inert to the biological/chemical materials into which the microtiter plate 102 comes in contact with. The raw materials can be polymerised, cross-linked and/or cured in the presence of a complementary shape, mould or die that are particularly well-suited for construction of the microtiter plate 102. Examples of suitable raw materials include urethane, latex, vinyl and silicone.

Under certain applications, such as fluorescence detection based assays, plastic materials that have low auto-fluorescence may be used to reduce the fluorescence noise which can interfere with the fluorescence from biological/chemical mixtures in the wells 110a, 110b, 110c. It is highlighted that the proposed embodiment contemplates for used in assays (or in preparation for performing assays) that may use fluorescence detection based methods. An example of such an assay is real-time quantitative PCR amplification of nucleic acid material. In an embodiment of such an assay, light from a source (which may have been filtered using a band-pass filter to provide light within a specific narrow range of wavelengths) enters the wells 110a, 110b, 110c, which are disposed with one or more biological/chemical materials that are sensitive to light of that range of wavelengths. The biological/chemical materials may fluoresce and emit light of a different range of wavelengths to that range of wavelengths to which the biological/chemical materials are sensitive. The emitted light (which may have been filtered using a band-pass filter to provide a narrow range of wavelengths) is detectable using a detecting means. The detecting means can be positioned within/external the microtiter plate 102. Accordingly, the microtiter plate 102 is constructed so as to allow light to enter the wells 110a, 110b, 110c. Further, the the microtiter plate 102 is constructed to allow light to enter the wells 110a, 110b, 110c and also exit the wells 110a, 110b, 110c through the cover 106. The cover 106 is formed to be substantially transparent to certain wavelengths of light. Glass can be used for the cover 106, wherein for example, the glass used has low-autofluorescence. An example of a fluorescence detection based assay is one that uses source light (that has been passed through a band-pass filter) of wavelengths in the range of 465 nm to 495 nm, and uses a detecting means capable of detecting emitted light (which has been passed through a band-pass filter) of wavelengths in the range of 515 nm to 555 nm.

In embodiments of the present invention, wherein a space 112 formed adjacent to the wells 110a, 110b, 110c is to be sealed with a substance (such as oil and a cured liquid pre-polymer), typically, the sealing substance also allows transmission of light into, and out of the wells 110a, 110b, 110c. Examples of plastics suitable for use to form the microtiter plate 102 include polypropylene (PP), polycarbonate (PC), polymethylmethacrylate (PMMA) and certain silicone materials. in particular the plastic for forming the microtiter plate 102 is polydimethylsiloxane (PDMS). Complementary moulds suitable for fabrication of components of the present invention, in particular the microtiter plate 102, may be made using a micro-machining technique. An example of such a technique is micro Electrical Discharge Machining (EDM) of steel plate and ICP etching of silicon wafer to form array of pillars that are used to replicate well array made of silicone and plastic materials by molding, casting, hot embossing, or made of metallic materials such as nickel by electroforming.

Yet further, it is however to be appreciated that the microtiter plate 102 can also be constructed using a mixture of different raw materials. In this respect, the properties of one raw material may lend themselves to that raw material being used to form certain components of the microtiter plate 102. Examples of properties that make a raw material suitable for use in a particular component the microtiter plate 102 include flexibility, surface functionality, hydrophilicity/hydrophobicity, ease of casting and cost of the raw material. Whilst certain raw materials may be selected to provide appropriate surface functionality for reaction with a substrate, all raw components are typically substantially inert to the chemicals/reaction mixtures with which they come into contact with. In particular, the raw materials used in the construction of devices and systems of the present invention will be compatible with the conditions of an intended application. For example, a technique of PCR requires efficient thermal transfer between a heat source/sink and each well 110a, 110b, 110c. Accordingly, for this PCR application, the raw materials used should typically be able to conduct heat efficiently and withstand thermal cycling without undergoing substantial deformation or melting. The properties of a given raw material can also be modified through selection of thickness etc. In these respects, PDMS represents a suitable material.

It is to be appreciated that the base of the microtiter plate 102 should be sufficiently thin and thermally conductive to facilitate fast thermal energy transfer between fluid in the wells 110a, 110b, 110c and a heating source such as a Peltier element that is in contact with the bottom of the microtiter plate 102. One example is that the base of the microtiter plate 102 comprises a thin well layer bottom and, optionally, an aluminum plate that is bonded to the bottom of the well layer (to be further described below). The base of the microtiter plate 102 is optionally attached to a flat and substantially rigid base member 105 (as shown in FIG. 2) for maintaining good thermal contact with the Peltier element during biological assay such as PCR thermal cycling.

Materials for forming the rigid base member 105 include metal (e.g. aluminium), glass, plastic and ceramic. Further, if the microtiter plate 102 is formed from two or more raw materials or layers of raw materials, various components of the microtiter plate 102 are attached together using a binding agent. For example, the binding agent used is applied in a substantially liquid form so as to bind the two components evenly across a surface, and subsequently undergoes a state transformation rendering the binding agent into solid state. An example of a method of application of such a binding agent is spin-coating. Where the microtiter plate 102 is made of glass and PDMS, the components may be attached together using liquid PDMS pre-polymer. In this regard, the curing of the PDMS pre-polymer forms a semi-permanent bond between the two components. In other embodiments, the base comprises a rigid layer of glass bonded with cured PDMS to a PDMS layer formed from a complementary mould wherein the PDMS layer comprises the array of wells 110.

In particular, a surface of the PDMS layer that is external to the opening of each well 110a, 110b, 110c is hydrophobic to avoid trapping any aqueous sample when removing the fluid sample 200 from the space 112. This is the same for surfaces of the cover 106 and the walls of the space 112. Moreover, those surfaces and walls are also to be compatible with biological assays.

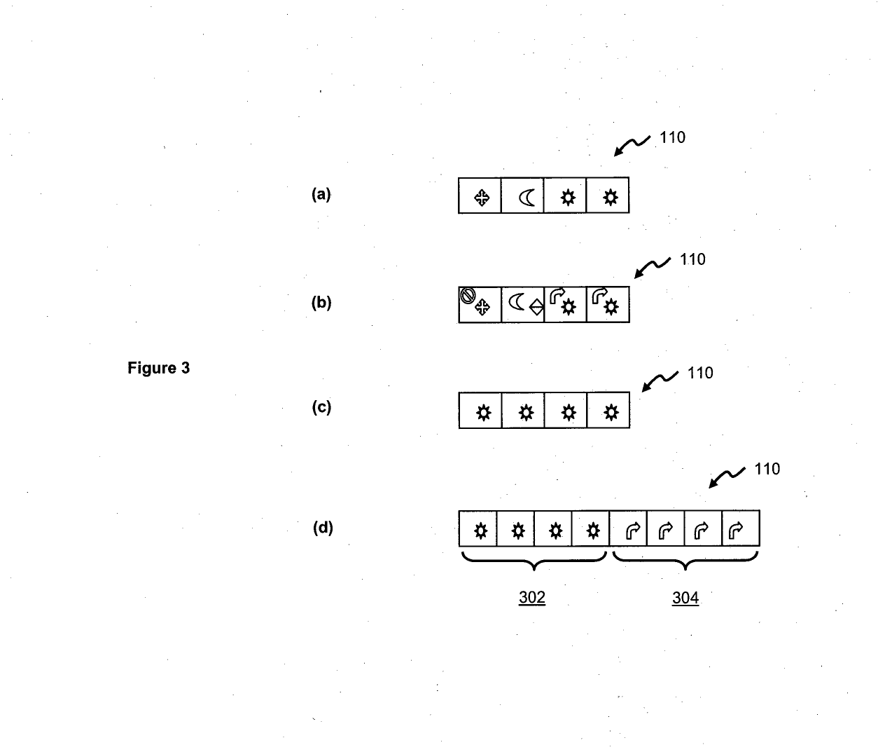

Depending on specific applications (e.g. polymerase chain reaction (PCR), immunoassays or the like) for the microfluidic device 100, the array of wells 110 can be preloaded with different biological/chemical materials. Preloading of the biological/chemical materials in the array of wells 110 can be carried out using a microarrayer machine or pipetting as known to persons skilled in the art. To illustrate, a few examples are shown in FIG. 3, and also for sake of discussion convenience hereinafter, primers are used as an example of the biological/chemical materials. It is however important to appreciate that the examples in FIG. 3 are not to be construed as limiting the types of biological/chemical materials that can be preloaded in the array of wells 110. For example, enzymes can also be used depending on an application. FIG. 3a shows that each well 110 is preloaded with a primer but of different types, whereas FIG. 3b depicts each well 110 being preloaded with multiple primers, in which the multiple primers loaded in the corresponding wells 110 are each of different types. On the other hand, the array of wells 110 can all be preloaded with a same type of primers as shown in FIG. 3c, whereas FIG. 3d shows that the array of wells 110 is divided into multiple zones (i.e. two zones 302, 304 in this instance), in which each zone 302, 304 is preloaded with the same type of primers. Further, in this instance, the array of wells 110 are collectively arranged as a square-like region in the central portion of the base, in which there are ten wells 110 arranged on each side of the square-like region, but it will be appreciated that other types and forms of arrangement are possible depending on an intended application for the microfluidic device 100. Typically, the array of wells 110 is arranged as a single one-dimensional layer in the base.

Still with reference to FIGS. 1 and 2, turning now to the cover 106, there is the space 112 formed at one surface, in which the space 112 has a shape and size that is complementary to the square-like region defined by the array of wells 110. More specifically, in this instance, the space 112 is arranged with a square-like shape and size to be similar with the square-like region defined by the array of wells 110. The space 112 also has an inlet channel 114 (at which an inlet control valve 116 is disposed adjacent thereto) and an outlet channel 118 (at which an outlet control valve 120 is disposed adjacent thereto). Particularly in this embodiment, the inlet channel 114 is for fluid to flow into the space 112 and the outlet channel 118 is for fluid to flow out of the space 112. It is also to be appreciated that the inlet channel 114 is formed to be wider than the outlet channel 118. Specifically, the outlet channel 118 leads to a container for collecting the fluid flowing out of the space 112. Thus the inlet control valve 116 is adjustable to allow fluid access to the space 112 and the outlet control valve 120 is adjustable to allow fluid to flow out of the space 112. Also, the inlet channel 114 is connected to a container comprising a reservoir of fluid, whereas the outlet channel 118 leads to another container for collecting fluid flowing out from the space 112, to be elaborated below. Alternatively, fluid flowing out from the outlet channel 118 may simply be discarded. In respective open positions, the inlet and outlet control valves 116, 120 enable regulation of flow of fluid into and out of the space 112 (as mentioned above), as well as an air pressure to be imposed within the space 112 (using the vacuum generating device 108), and thus conveniently positioned on an external surface of the cover 106 to facilitate manual adjustments to be made easily (e.g. by an operator of the microfluidic device 100). Conversely, in respective closed positions, the inlet and outlet control valves 116, 120 do not enable fluid to flow into/out of the space 112, as will be apparent to skilled persons. In summary, the inlet and outlet control valves 116, 120 (respectively disposed at the inlet and outlet channels 114, 118) control fluid access to the space 112; i.e. in the open positions, the inlet and outlet control valves 116, 120 allow access to the space 112, whereas in the closed positions, the inlet and outlet control valves 116, 120 deny access to the space 112.

To assemble the microfluidic device 100, the surface of the cover 106, at which the space 112 is formed, is fitted over the base of the microtiter plate 102 and is aligned such that the space 112 is positioned directly adjacent to the array of wells 110. In this instance, the space 112 is arranged above the array of wells 110. It is thus to be appreciated that the space 112 above the array of wells 110 is defined by the cover 106, which acts as a cover fitting over the microtiter plate 102. It will also be apparent that in this instance, the space 112 above the array of wells 110 is positioned between the inlet and outlet channels 114, 118.

Thereafter, the cover 106 and the base are securely attached to each other in a manner to support a differential pressure environment therewithin. In this embodiment, the flat and substantially rigid base member 105 is removably attached to the base of the microtiter plate 102 to prevent warping of the base that can occur when an air pressure subsequently formed within the space 112 is lower than atmospheric pressure. An example of a material suitable for use as the rigid base member 105 is aluminium, as aluminium allows for efficient heat transfer, which is important to certain applications of the microfluidic device 100 such as for nucleic acid amplification techniques (e.g. PCR). It is to be appreciated that any sort of pressure referred to in this specification refers to fluid pressure. Particularly, a differential pressure is to be subsequently generated (when required) using the vacuum generating device 108 for controlling speed of flow of the fluid sample 200 or a sealant 202 through the space 112, which will be elaborated below. It is also to be appreciated that space 112 thus forms a headspace above the array of wells 110, and as a result, the space 112, the inlet channel 114, the outlet channel 118 and the array of wells 110 are in fluid communication with one another. Moreover, in this arrangement, openings of the respective wells 110 directly face and connect with the space 112, which advantageously allows any air pockets trapped within the associated wells 110 to be released into the space 112 during device operation and accordingly improves performance reliability of the microfluidic device 100. Correspondingly, the array of wells 110 are said to be configured in an open wells arrangement.

Referring now to the vacuum generating device 108, the first vacuum generator 1081 is equipped with a chamber 1081a for holding the fluid sample 200 and/or the sealant 202, an inlet tubing 1081b and an air inlet 1081c. An air pump 1202, with an associated air pump valve 1204, for generating atmospheric (or higher than atmospheric) pressure is also coupled to the chamber 1081a via the air inlet 1081c (at an attachment port 1206). It is to be understood that the chamber 1081a is the container, comprising the reservoir of fluid (i.e. the fluid sample 200/sealant 202) as afore described, to which the inlet channel 114 is connected. It is also appreciated that while, in this embodiment, the fluid sample 200 and sealant 202 are held in the same common container, the sealant may however be held in another separate container to the fluid sample 200 (i.e. see second embodiment below). One end of the inlet tubing 1081b is detachably attached to the inlet channel 114 of the space 112, while the other end extends substantially into and along the length (and towards the bottom) of the chamber 1081a of the first vacuum generator 1081. In addition, the air inlet 1081c of the first vacuum generator 1081 is in turn coupled to a first vacuum source (not shown) via an air port 1081d which is configured with a corresponding pressure regulator 1081e. An example of the first vacuum source is a vacuum pump. Further, the first vacuum generator 1081 is arranged with an air intake port 1081f (having an associated valve), which connects directly to the air inlet 1081c. That is, the air intake port 1081f bypasses control of the pressure regulator 1081e, and is thus located at an end in opposition to where the air port 1081d is. The air intake port 1081f is particularly configured to allow air at atmospheric pressure to be introduced into the chamber 1081a of the first vacuum generator 1081 when the associated valve is opened, and vice versa when the valve is closed. The pressure regulator 1081e enables adjustment of a desired air pressure applied to the chamber 1081a of the first vacuum generator 1081 via the first vacuum pump. The transfer of the fluid sample 200 or sealant 202 into/out of the space 112 is accomplished by utilising a difference in level of pressure configured between the chamber 1081a of the first vacuum generator 1081 and the space 112 or the array of wells 110, or can also be done by applying compressed air to the chamber 1081a of the first vacuum generator 1081 to drive the fluid sample 200 or sealant 202 held in the chamber 1081a into the space 112 through the inlet tubing 1081b.

Examples of the fluid sample 200 include a sample containing nucleic acid molecules (e.g. DNA, RNA, mRNA, microRNA, cDNA etc), cells, Taq polymerase enzyme for PCR, fluorescent probes, solid particles such as fluorescent particles, fluorescent dye molecules/chemicals or the like. On the other hand, the sealant 202 is typically a liquid immiscible in and less dense than the fluid sample 200, and suitable for forming a liquid seal adjacent to and covering the wells 110a, 110b, 110c that have been filled with the fluid sample 200 (by completely covering the openings to the filled wells 110a, 110b, 110c), but without entering those wells 110a, 110b, 110c (through mixing with the fluid sample 200). It will thus be appreciated that when the fluid sample 200 and sealant 202 are held together in the chamber 1081a of the first vacuum generator, two clear separating fluid layers can be seen due to the immiscible property. In addition, a liquid used as the sealant 202 should not significantly inhibit chemical or biochemical analysis of the fluid sample 200, for example, using PCR thermal cycling. The sealant 202 also needs to be transparent and has low auto-fluorescence to allow fluorescence emitted from the preloaded biological/chemical materials (that are suspended in the fluid sample 200) in the wells 110a, 110b, 110c to reach an external optical detection device (not shown) with low background optical noise. Examples of liquid that can be used as the sealant 202 include oil, polymer resins, silicone pre-polymer and the like. The sealant 202 can also be a curable liquid polymer (i.e. thermal curable or UV curable), which in the cured state, forms a solid sealant in the space 112. Specific usage of the fluid sample 200 and sealant 202 will be further set out in subsequent description below in a corresponding method regarding use of the microfluidic device 100.

Similarly, the second vacuum generator 1082 is also correspondingly equipped with a chamber 1082a for holding the fluid sample 200 and/or the sealant 202, an outlet tubing 1082b and an air inlet 1082c. It is to be understood that the chamber 1082a is the container, as afore described, to which the fluid (i.e. the fluid sample 200/sealant 202) is collected after flowing out from the space 112 via the outlet channel 118. One end of the outlet tubing 1082b is detachably attached to the outlet channel 118 of the space 112, while the other end extends substantially into and along the length of the chamber 1082a of the second vacuum generator 1082. Further, the air inlet 1082c of the second vacuum generator 1082 is in turn coupled to a second vacuum source (not shown) via an air port 1082d which is configured with a corresponding pressure regulator 1082e. An example of the second vacuum source is a vacuum pump. Further, the second vacuum generator 1082 is arranged with an air intake port 1082f (having an associated valve), which connects directly to the corresponding air inlet 1082c. That is, the air intake port 1082f (i.e. refer to FIG. 2) bypasses control of the associated pressure regulator 1082e, and is thus located at an end in opposition to where the air port 1082d of the second vacuum generator 1082 is. The air intake port 1082f is particularly configured to allow air at atmospheric pressure to be introduced into the chamber 1082a of the second vacuum generator 1082 when the associated valve is opened; and vice versa when the valve is closed. As before, the pressure regulator 1082e in this instance enables adjustment of a desired air pressure applied to the chamber 1082a of the second vacuum generator 1082 via the second vacuum pump. It is also to be appreciated that the inlet and outlet tubings 1081b, 1082b used are adopted from typical standard tubings (e.g. made of silicone, plastic, metal or the like) as will be understood by skilled persons.

Importantly, the first and second vacuum generators 1081, 1082 are in cooperative arrangement to enable a differential pressure (as appropriate) to be generated within the space 112 and the wells 110a, 110b, 110c. Particularly, the cooperative arrangement between the first and second vacuum generators 1081, 1082 for generating a differential pressure is achieved through coordinated adjustment of the respective pressure regulators 1081e, 1082e in order to control the speed of flow of the fluid sample 200/sealant 202 through the space 112. And more specifically, by coordinating the adjustment of the respective pressure regulators 1081e, 1082e, the first and second vacuum generators 1081, 1082 are each then operated to generate different absolute pressure to facilitate generation of the differential pressure to control a rate and speed of flow of the fluid sample 200/sealant 202 into the space 112. Specifically in this respect, the first vacuum generator 1081 is configured to generate a first absolute pressure in the vicinity of the inlet channel 114, while the second vacuum generator 1082 is configured to generate a second different absolute pressure in the vicinity of the outlet channel 118. It is to be understood that “vicinity of the inlet channel 114” means in close proximity to the inlet channel 114 and may also mean within the inlet channel 114. Similarly, “vicinity of the outlet channel 118” means in close proximity to the outlet channel 118 and may also mean within the outlet channel 118. Further, it is also important to appreciate that in using the first and second vacuum generators 1081, 1082 and/or inlet and outlet control valves 116, 120, the differential pressure can be adjusted to precisely control the rate of fluid flow within the microfluidic device 100, such that the fluid flow may even be stopped, if required.

The microfluidic device 100 also has a fluid flow sensor 204 that is disposed external to the cover 106, and more specifically, in a position substantially adjacent to the inlet control valve 116 in order to determine an approaching flow of fluid sample 200/sealant 202 entering/leaving the space 112 through the inlet channel 114. The fluid flow sensor 204 operates by detecting a change of refractive index within the inlet channel 114. In this instance, a change of refractive index within the inlet channel 114 occurs and is detected, when the space within the inlet channel 114 is replaced by the approaching flow of the fluid sample 200/sealant 202 entering the inlet channel 114. Further, it will also be appreciated by now that independently adjusting the inlet and outlet control valves 116, 120 enables/denies fluid communication between the space 112 and the wells 110a, 110b, 110c, and the respective chambers 1081a, 1082a of the first and second vacuum generators 1081, 1082.

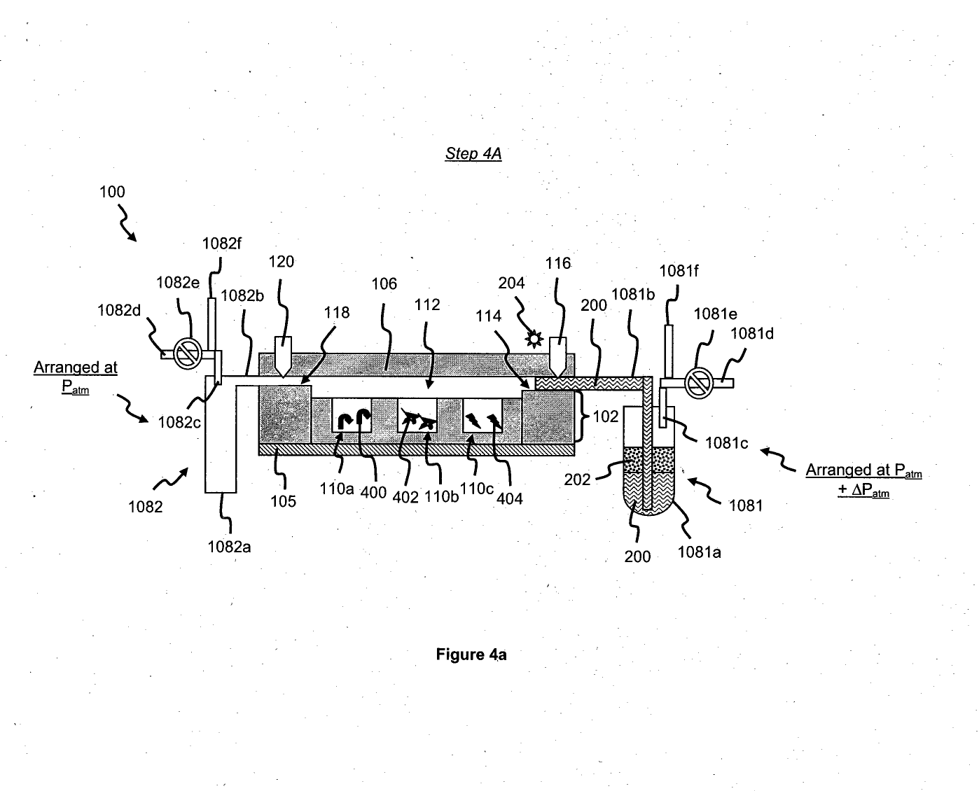

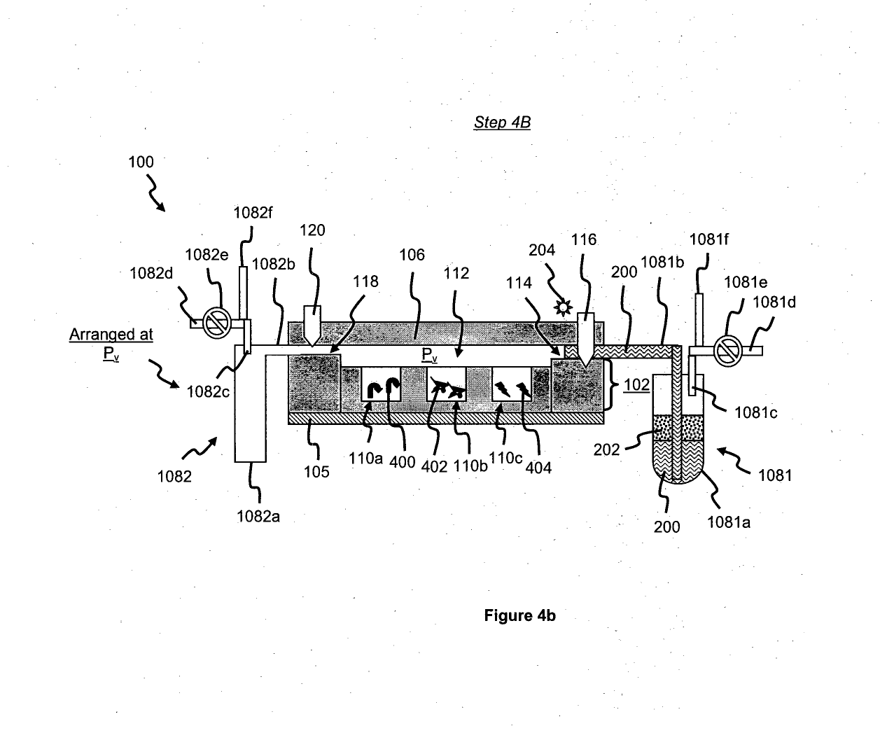

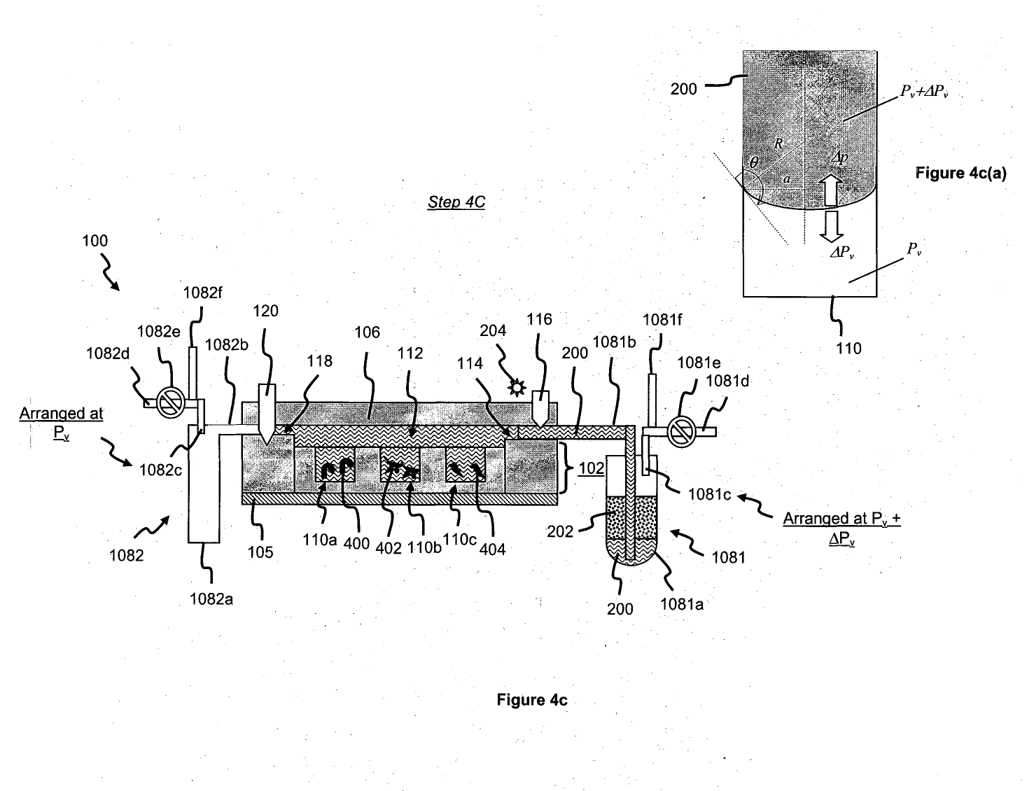

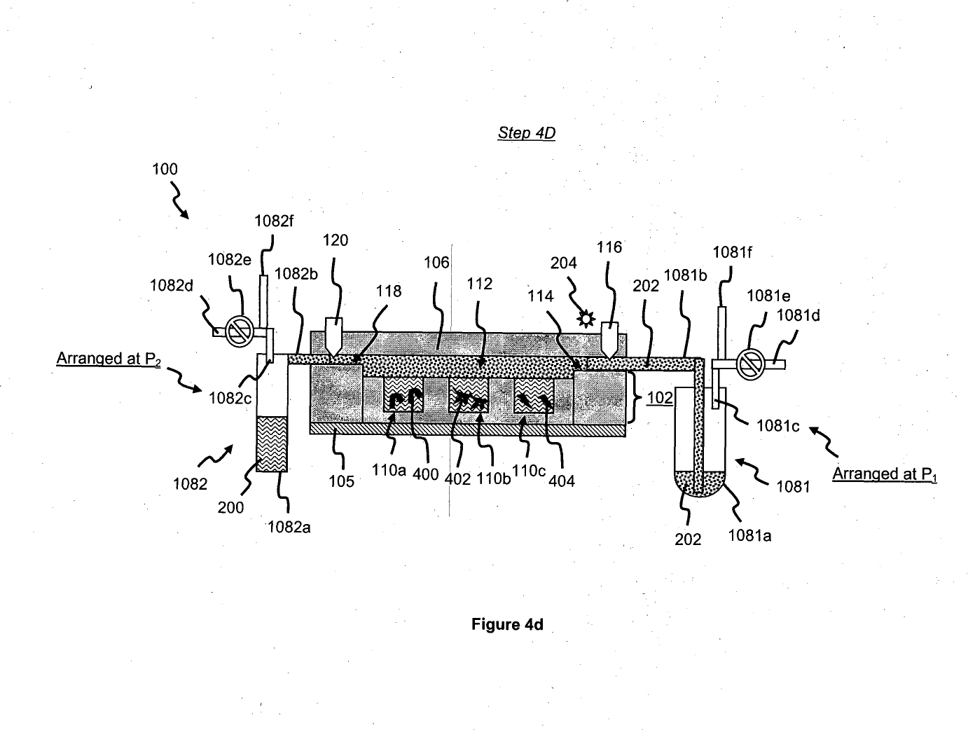

According to the embodiment, FIGS. 4a to 4d collectively illustrate a method for using the microfluidic device 100. In particular, the method comprises Steps 4A to 4D, and relates to introducing the fluid sample 200 into the space 112 to fill the wells 110a, 110b, 110c (which in this instance are respectively preloaded with different types of primers 400, 402, 404) and thereafter sealing the filled wells 110a, 110b, 110c with the sealant 202, so that PCR thermal cycling (for example) can be carried out without requiring the fluid sample 200 to be first evaporated from the wells 110a, 110b, 110c. In Step 4A as shown in FIG. 4a, the wells 110a, 110b, 110c are first preloaded with the different types of primers 400, 402, 404, as desired, based on an intended usage of the microfluidic device 100. The chamber 1081a of the first vacuum generator 1081 is then preloaded with both the fluid sample 200 and sealant 202, in which the sealant 202 floats on the fluid sample 200 (i.e. the fluid sample has a heavier fluid density than the sealant 202). In Step 4A, both the inlet and outlet control valves 116, 120 are arranged in the open positions and a first absolute pressure at atmospheric level (i.e. Pₐₜₘ) is applied to the air inlet 1082c of the second vacuum generator 1082 by opening the associated air inlet 1082c to ambient pressure. Further, a second absolute pressure, being slightly higher than atmospheric level (i.e. Pₐₜₘ+ΔPₐₜₘ), is on the other hand applied using the air pump 1202 via the air intake port 1081f of the first vacuum generator 1082, to the air inlet 1081c of the first vacuum generator 1081. As aforementioned, the air pump 1202 is coupled to the air inlet 1081c of the first vacuum generator 1081 through the attachment port 1206. It will also be appreciated that the second absolute pressure is higher than the first absolute pressure, and is adjustable independent of the first absolute pressure. As a result, the fluid sample 200 is driven, by the higher air pressure, out from the chamber 1081a of the first vacuum generator 1081 into the inlet tubing 1081b, and then into the inlet channel 114 of the space 112, stopping at a position subsequent to the inlet control valve 116, but prior to entering the space 112. It is to be noted that the position within the inlet channel 114 of the space 112, at which the flow of the fluid sample 200 is stopped, is determined using the fluid flow sensor 204 or a visualization means such as a camera or human eyes. The purpose of this is that it beneficially prevents possible formation of an air column between the inlet control valve 116 and a fluid front of the fluid sample 200, in case the fluid front does not flow to a position at where the inlet control valve 116 is located, which can subsequently cause a vacuum that is to be generated in Step 4C of this method to be of an insufficient pressure.

Further next, in Step 4B, the inlet control valve 116 is now switched to the closed position, with the outlet control valve 120 still maintained in the open position, and consequently this allows a vacuum pressure, Pᵥ, in the space 112 to be reduced to approximately between 10⁻⁸ torr to 700 torr through making of appropriate adjustments through the air inlet 1082c of the second vacuum generator 1082. It will be appreciated that 1 bar is equivalent to 100 kPa, 1000 millibar, 750 mmHG, or 750 torr. Further atmospheric pressure (i.e. ambient pressure) is defined to be at approximately 101.3 kPa. This is specifically achieved using the second vacuum generator 1082 in which the associated pressure regulator 1082e of the second vacuum generator 1082 is adjusted in order for the vacuum pressure of Pᵥ to be attained within the space 112 at Step 4B. Thus, the first absolute pressure is changed to Pᵥ. It is to be appreciated that Pᵥ is a reduced pressure relative to atmospheric pressure.

Subsequently in Step 4C, the outlet control valve 120 is then switched to the closed position after a vacuum pressure in the space 112 has been adjusted to reach the value of Pᵥ through the air inlet 1082c of the second vacuum generator 1082, as performed in the afore Step 4B. It is to be highlighted that the outlet control valve 120 can be closed because the vacuum pressure in the space 112 is configured to be sufficiently high (i.e. greater or equal to approximately between 10⁻⁸ torr to 700 torr). Specifically, this prevents possible movement of the fluid sample 200 to flow through and out of the space 112, which may cause excessive loss of sample and possibly contamination of second vacuum generator 1082. Therefore, the space 112 when arranged in this manner is said to be in a closed-end headspace arrangement.

In addition, with the vacuum pressure of Pᵥ being maintained at the air inlet 1082c of the second vacuum generator 1082, a slightly higher vacuum pressure of Pᵥ+ΔPᵥ is now applied to the air inlet 1081c of the first vacuum generator 1081, which is achieved by applying a vacuum pressure of equal to or lower than Pᵥ+ΔPᵥ at the air port 1081d of the first vacuum generator 1081 and adjusting a vacuum pressure at the air inlet 1081c of the first vacuum generator 1081 to Pᵥ ΔPᵥ by using the associated pressure regulator 1081e. In other words, the second absolute pressure is now changed to Pᵥ+ΔPᵥ (using the first vacuum generator 1081), while the first absolute pressure is still at Pᵥ. It is to be appreciated that Pᵥ+ΔPᵥ is a reduced pressure relative to atmospheric pressure, and the first and second absolute pressures are adjustable independent of each other. Moreover, it is to be appreciated that ΔPᵥ represents a difference in vacuum pressure between the air inlet 1081c of the first vacuum generator 1081 and the space 112. That is, the absolute pressure level Pᵥ is lower than that of Pᵥ+ΔPᵥ, resulting in a differential pressure, to cause the fluid sample 200 to be driven into the space 112 when the inlet control valve 116 is subsequently opened. More importantly, it is to be highlighted that ΔPᵥ is set to an appropriate (small) value suitable for controllably driving the fluid sample 200 (as well as the sealant 202) into the space 112 and the wells 110a, 110b, 110c at a desired speed (which can be substantially slow or fast, as necessary). In other words, by virtue of different values of ΔPᵥ, the fluid sample 200 can be driven into the space 112 at different controllable speeds, independent of the value of Pᵥ. For example, to drive the fluid sample 200 to flow in the space 112 at a speed of approximately between 1 μm/second to 100 mm/second, the value of ΔPᵥ is to be defined at approximately between 0.01% of Pᵥ to 100% of Pᵥ. As a comparison, in conventional devices operating using only a single vacuum configuration, wherein the first absolute pressure is arranged to be at 10 torr, and the second absolute pressure is arranged to be at atmospheric pressure, a speed of flow of the fluid sample 200 through the space 112 will be around 750 mm/second, which is undesirably high compared to the current embodiment. It will be appreciated that this method enables the fluid sample 200 to flow at a speed (determined solely by ΔPᵥ) independently from a desired value of Pᵥ in the space 112 and wells 110a, 110b, 110c.

Thereafter, the inlet control valve 116 is open to allow the fluid sample 200 to move at a controllable slow speed from the chamber 1081a of the first vacuum generator 1081 into the space 112 and the wells 110a, 110b, 110c to completely fill the space 112 and the wells 110a, 110b, 110c with the fluid sample 200. In particular, the fluid sample 200 is driven into the space 112 at the slow speed until the fluid sample is stopped by the closed outlet control valve 120 or by closing the outlet control valve 120, as shown in Step 4C. Thus, the fluid sample 200 flows from the inlet channel 114 to the outlet channel 118 due to the resulting differential pressure. It is to be highlighted that the slow speed at which the fluid sample 200 is moving through the space 112 (and into the wells 110a, 110b, 110c) beneficially prevents the preloaded primers 400, 402, 404 (that are mixed/re-suspended with the fluid sample 200 once the wells 110a, 110b, 110c are filled with the fluid sample 200) from being flushed out of the respective wells 110 into the space 112 and undesirably cross-contaminating neighbouring wells 110a, 110b, 110c. It is important to appreciate that the aforementioned cross-contamination of the neighbouring wells 110a, 110b, 110c can occur if a speed of flow of the fluid sample 200 is relatively high, which will consequently generate high shear stress forces or/and impingement to pull (any one of) the preloaded primers 400, 402, 404 out of the associated wells 110a, 110b, 110c.

The choice of ΔPᵥ depends on many factors, including well size, well geometry such as presence of sharp corners at the well bottom, depth of well, time for material at the bottom to travel to well opening, location of the preloaded material deposition in the well, amount of preloaded material in the well, tolerance of assay in losing the material, tolerance of assay in cross-contaminated well, etc.

Firstly, the vacuum pressure differential ΔPᵥ needs to be large enough to push the sample into the well and fill as much space as possible to interact with pre-loaded material over the well surface and to minimize air bubble formation during assay. When the sample liquid enters the well, it encounters the capillary forces induced on the well surface. Depending on the surface energy of the well surface and the sample and many other factors, the well surface can behave like a hydrophobic or hydrophilic surface. In physics, the Young-Laplace equation is used to describe the capillary pressure difference sustained across the interface between two static fluids, such as water and air, due to the phenomenon of surface tension or wall tension. It is a statement of normal stress balance for static fluids meeting at an interface, where the interface is treated as a surface:

$$\begin{matrix}Δ p = - γ ∇ \cdot \hat{n} \\ = 2 γ H \\ = γ ( \frac{1}{{R}_{1}} + \frac{1}{{R}_{2}} )\end{matrix}$$

where Δₚ is the pressure difference across the fluid interface, γ is the surface tension (or wall tension), \hat{n} is the unit normal pointing out of the surface, H is the mean curvature, and R₁ and R₂ are the principal radii of curvature. In a sufficiently narrow tube or a well 110 of circular cross-section (radius a), as shown in FIG. 4c(a), the interface between sample 200 and air (vacuumed) in the well 110 forms a meniscus that is a portion of the surface of a sphere with radius R. The above pressure jump across this surface, or capillary pressure, becomes:

$$Δ p = \begin{matrix}2 γ \\ R\end{matrix} .$$

The radius of the sphere R is a function only of the contact angle, θ, which in turn depends on the exact properties of the fluids and the solids in which they are in contact:

$$R = \frac{a}{cos θ}$$

so that the pressure difference may be written as:

Δp=(2γ cos θ/a)

For a water-based sample 200, if the well surface is hydrophobic, the contact angle larger than 90° (refer to FIG. 4c(a) for the case of hydrophobic surface), and if the well surface is hydrophilic, the contact angle less than 90°. As shown in FIG. 4c(a), in order to maintain hydrostatic equilibrium, the induced capillary pressure Δp is balanced by the vacuum pressure differential ΔPᵥ which can be positive (pointing downward) or negative (pointing upward), depending on whether the wetting angle is less than or greater than 90°. Therefore, such hydrostatic equilibrium gives,

ΔPᵥ=(2γ cos θ)/a,

from which it can conclude that the smaller well 110 or a cavity in the well 110 to fill with the sample 200, the larger the vacuum pressure differential ΔPᵥ is needed to provide. In this regard, a higher ΔPᵥ is desirable in minimize air bubble size which may form during assay.

On the other hand, the higher the ΔPᵥ, the higher the flow speed when the sample enters the well. Also, after the well is filled with the sample, the sample still flows forward outside the well opening, generating a shear induced vortex inside the well. The vortex strength is proportional to the sample flow speed over the well opening. The vortex can cause a recirculation of a flow field inside the well that is able to transport the pre-loaded material near the well surface including the well bottom to the well opening area, and the mass transfer by convection and/or diffusion at the well opening area can move the pre-loaded material into the space outside the well, causing the loss of pre-loaded material and cross-contamination of adjacent wells. Therefore, ΔPᵥ cannot be too high in order to minimize the loss of pre-loaded material in the well, and such considerations to choose a ΔPᵥ value depend on dimensions of the well which affect the size of the well opening and depth of the well (related to time for material at the bottom to travel to well opening), location of the preloaded material deposition in the well, amount of preloaded material in the well, tolerance of assay in losing the material, tolerance of assay in cross-contaminated well. In general, ΔPᵥ has to be larger than a critical value obtained from minimizing the air pocket volume in the wells under vacuum, and smaller than a critical value obtained from having a sufficiently low sample speed to minimize the flushing of the pre-loaded materials in the wells.

Another method to fill the small cavity in the well while maintaining a small ΔPᵥ to minimizing flushing the sample 200 in the well 110 is to apply a small ΔPᵥ to achieve a low speed of sample loading, and after completion of the sample loading into the wells 110, a sufficiently high ΔPᵥ is applied to push the sample 200 into any voids at the bottom of the wells 110. This method is similar to that shown in FIG. 4D in which a sufficient compressed air pressure P1 and/or P2 are applied in the sealant loading step (FIG. 4D) which will be described in a later paragraph.

In last Step 4D of the method, once the fluid sample 200 fills the wells 110a, 110b, 110c and the space 112, the air inlet 1081c of the first vacuum generator 1081 (as well as the sealant 202) is applied with an air pressure of P₁ at a first vacuum level and the air inlet 1082c of the second vacuum generator 1082 is applied with another air pressure of P₂ at a second vacuum level. It is to be appreciated that the air pressures of P₁ is higher than P₂, resulting in a differential pressure, in order that the sealant 202 can be driven into the space 112 and thereafter fill the space 112 when the inlet and outlet control valves 116, 120 are switched to the open positions. In this instance, the air pressure of P₁ is defined to be at the vacuum pressure of Pᵥ+ΔPᵥ as afore applied to the air inlet 1081c of the first vacuum generator 1081 at Step 4C, and the air pressure of P₂ is defined to be at the vacuum pressure of Pᵥ as afore applied to the air inlet 1082c of the second vacuum generator 1082 at Step 4C. It is to be appreciated that the higher a difference between the air pressures of P₁ and P₂, the higher a speed of flow of the sealant 202 in the space 112. Specifically, the difference between the air pressures of P₁ and P₂ is to be controlled below a threshold value that enables flow of the sealant 202 to be sufficiently slow to prevent generation of a high shear stress forces at a fluid interface formed between the sealant 202 and the fluid sample 200 in the wells 110a, 110b, 110c (exposed through the associated openings of the wells 110a, 110b, 110c) that would otherwise drag the fluid sample 200 (together with the preloaded primers 400, 402, 404) out and emptying the associated wells 110a, 110b, 110c and also further causes the sealant 202 to flow into the emptied wells 110a, 110b, 110c as a result. It will be appreciated that pressured or compressed air can also be used as the air pressures of P₁ and P₂. One of the benefits of using the compressed air for P₁ and P₂ is that the high pressure P₁ and P₂ can press the fluid sample 200 filled in the wells 110a, 110b, 110c further down to fill any small cavities or sharp corners over the well surface which can form air bubble nucleation sites during thermal cycling in later stages of sample analysis. It is to be appreciated that references to the fluid sample 200 in the preceding sentence also include references to the sealant 202 under the appropriate context. It will also be appreciated that a flow speed of the sealant 202 through the space 112 can be controlled independently as well, similar in a manner to that for the fluid sample 200 as afore described at Step 4C.

Accordingly, once the air inlet 1081c of the first vacuum generator 1081 and the air inlet 1082c of the second vacuum generator 1082 are applied respectively with the air pressures of P₁ and P₂, the resulting differential pressure as generated then drives the sealant 202 (as well as any remaining fluid sample 200) from the chamber 1081a of the first vacuum generator 1081 into the space 112, upon switching the outlet control valve 120 to the open position. This consequently pushes the fluid sample 200 residing in the space 112 out into the chamber 1082a of the second vacuum generator 1082 to be temporarily stored. It is to be appreciated that during the process, the fluid sample 200 together with the preloaded primers 400, 402, 404 that are already in the wells 110a, 110b, 110c remain within the associated wells 110a, 110b, 110c and are not pushed out by the sealant 202. The sealant 202 further subsequently fills and completely occupies the entire space 112 which has an effect of sealing the wells 110a, 110b, 110c. Thus, in this embodiment, the sealant 202 is introduced into the space 112 to remove the fluid sample 200 subsequent to filling the wells 110a, 110b, 110c with the fluid sample 200 and thereafter, the space 112 is then filled with the sealant 202 to seal the wells 110a, 110b, 110c already filled with the fluid sample 200. Any excess sealant 202 in the space 112 will then also overflow into the chamber 1082a of the second vacuum generator 1082. It is also to be appreciated that the vacuum pressure difference of ΔPᵥ is important in maintaining a slow speed of flow of the sealant 202 in the space 112 to beneficially prevent the sealant 202 from disintegrating into blobs that can otherwise result in possible mixing with the fluid sample 200 flowing in front of the sealant 202 (i.e. referenced in the context of a direction towards the outlet channel 118 of the space 112) in the space 112. When such mixing of the fluid sample 200 with disintegrated blobs of the sealant 202 occurs, it can result in the sealant 202 flowing into the wells 110 and/or being unable to effectively purge the fluid sample 200 out of the space 112 into the chamber 1082a of the second vacuum generator 1082, as originally intended.

Further embodiments of the invention will be described hereinafter. For the sake of brevity, description of like elements, functionalities and operations that are common between the embodiments are not repeated; reference will instead be made to similar parts of the relevant embodiment(s).

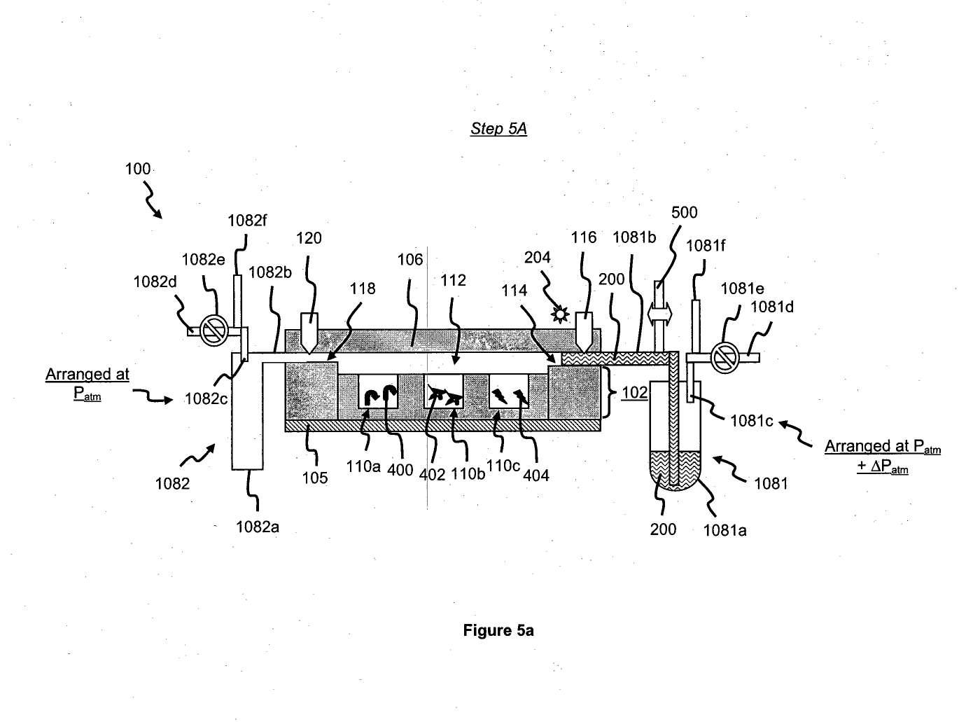

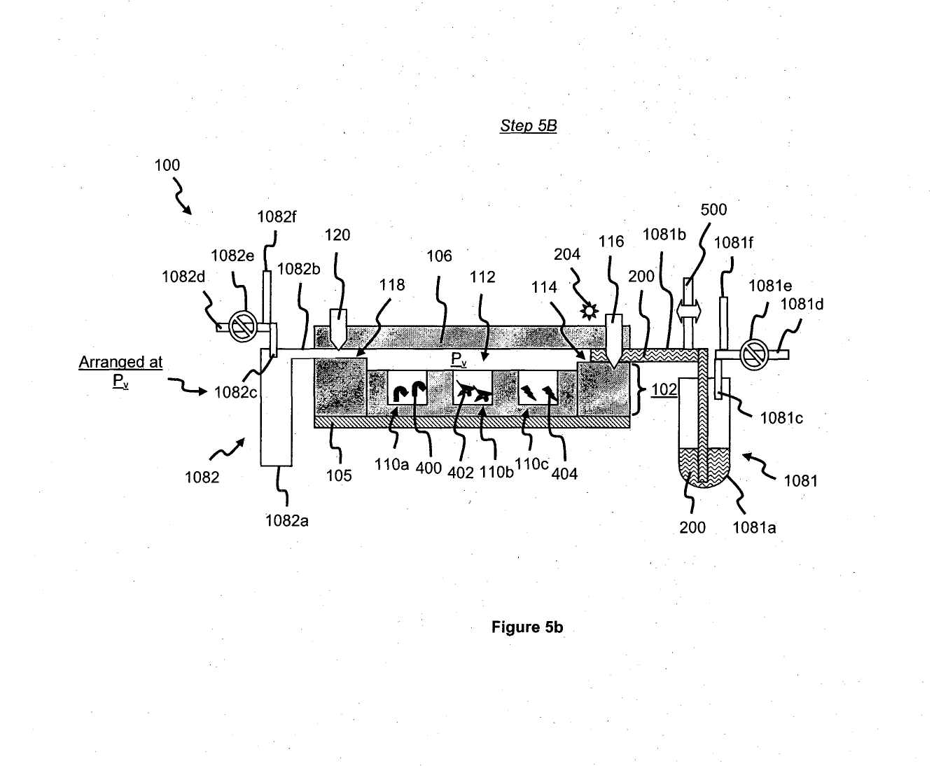

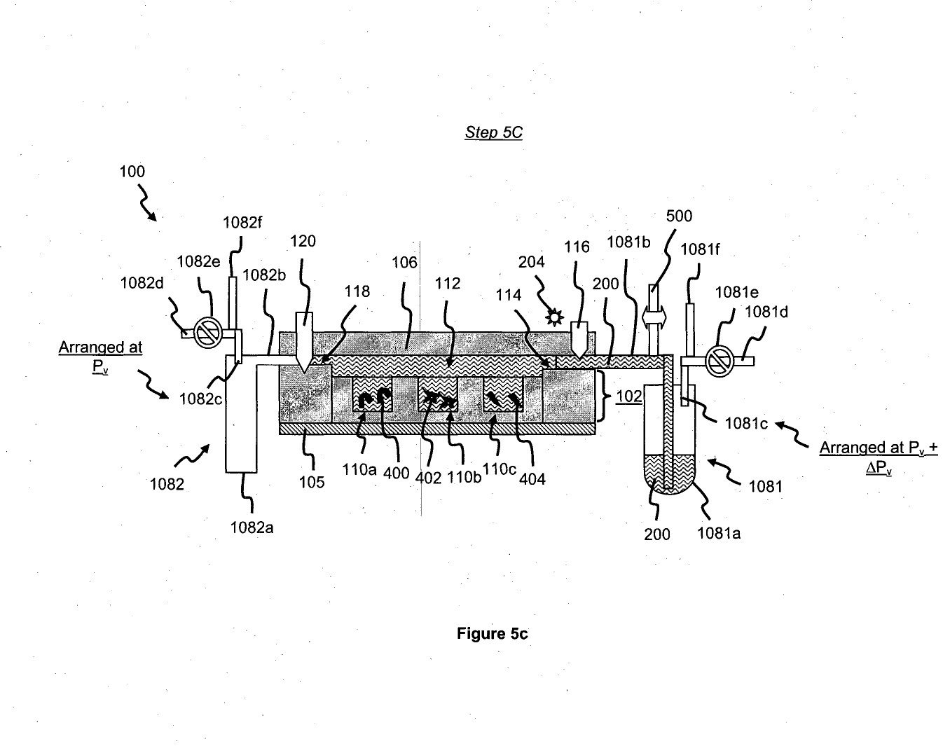

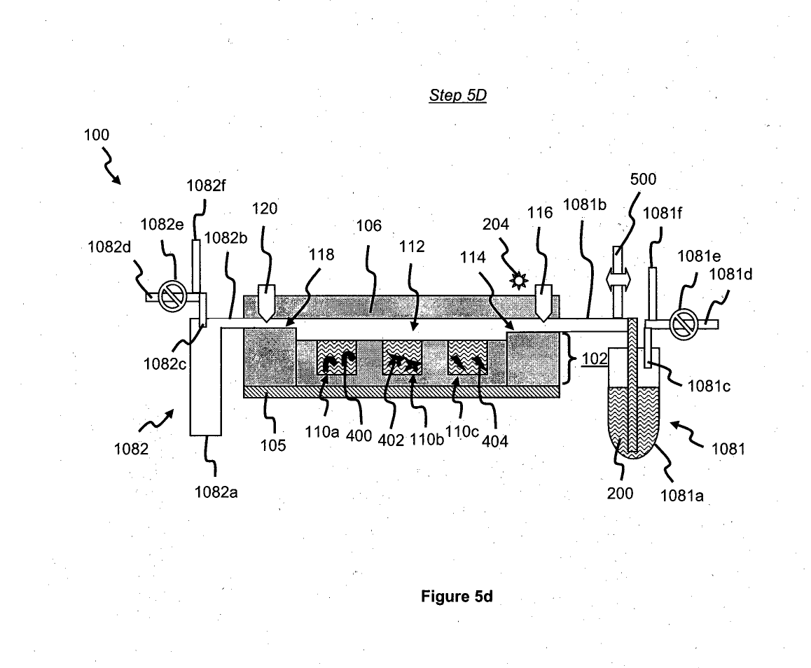

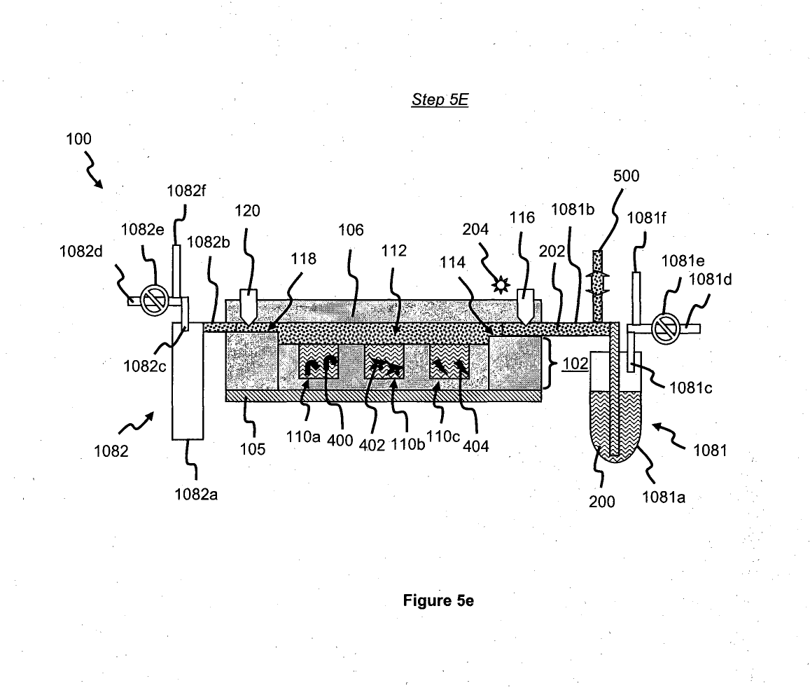

According to a second embodiment, FIGS. 5a to 5e collectively illustrate another method, comprising Steps 5A to 5E, for introducing the fluid sample 200 into the space 112 to fill the wells 110a, 110b, 110c (which are respectively preloaded with the different types of primers 400, 402, 404) and thereafter sealing the filled wells 110a, 110b, 110c with the sealant 202. It is to be highlighted that the microfluidic device 100 in this instance is further equipped with an additional channel 500 that is further connected to the inlet tubing 1081b of the first vacuum generator 1081, but is otherwise similar to that as described in the first embodiment. Specifically, the additional channel 500 is for introducing the sealant 202 into the space 112, instead of introducing the sealant 202 via the chamber 1081a of the first vacuum generator 1081, as in afore described of FIGS. 4a to 4d. Therefore, it will be apparent that in this embodiment, the sealant 202 is not held in the chamber 1081a of the first vacuum generator 1081, but rather in an external sealant dispenser (not shown) that holds only the sealant 202. That said, Steps 5A to 5C (shown in FIGS. 5a to 5c) are carried out in the same manner as the Steps 4A to 4C of FIGS. 4a to 4c, and therefore for sake of brevity, will not be repeated herein.

In next Step 5D as shown in FIG. 5d, the outlet control valve 120 is switched to the open position from its closed position in Step 5C. Then, before introducing the sealant 202 into the space 112, the fluid sample 200 is first removed from the space 112 by withdrawing the fluid sample 200 into the chamber 1081a of the first vacuum generator 1081 or pushing the fluid sample 200 into the chamber 1082a of the second vacuum generator 1082. Particularly, an air pressure of slightly lower than atmospheric pressure is applied to the air port 1081d of the first vacuum generator 1081 to withdraw the fluid sample 200 into the chamber 1081a of the first vacuum generator 1081, or alternatively an air pressure of slightly higher than atmospheric pressure is applied to the air port 1081d of the first vacuum generator 1081 to push the fluid sample 200 into the chamber 1082a of the second vacuum generator 1082. As an example in this instance, the fluid sample 200 is withdrawn into the chamber 1081a of the first vacuum generator 1081. In last Step 5E of the method, the sealant 202 is introduced via the additional channel 500 into the space 112 to completely fill and occupy the space 112. As will be appreciated, this has an effect of sealing the wells 110a, 110b, 110c, similar to the method of the first embodiment.

Hence, in this embodiment, the fluid sample 200 is removed from the space 112 subsequent to filling the wells 110a, 110b, 110c with the fluid sample 200, and thereafter, the sealant 202 is introduced into the space 112 to seal the wells 110a, 110b, 110c already filled with the fluid sample 200. Any excess sealant 202 in the space 112 will then overflow into the chamber 1081a of the first vacuum generator 1081 or the chamber 1082a of the second vacuum generator 1082.

According to a third embodiment, all the respective Steps 5A to 5C and 5E remain the same as in the second embodiment, with a difference effected only in Step 5D. More specifically, in Step 5D of this current embodiment, the fluid sample 200 is removed from the space 112 by first changing an orientation of the space 112 which can be achieved by tilting the assembled portion comprising the cover 106 and microtiter plate 102 at any desired angle, with respect to a resting base on which the microfluidic device 100 is disposed. For example, the space 112 is orientated substantially perpendicular to the resting base for optimal results. Thereafter, vacuum or air pressure or capillary force via an absorbent, is applied in the manner as described in afore embodiments, to draw the fluid sample 200 out from the space 112, in conjunction with assistance using centrifugal force or gravity, which is effected due to the space 112 being in a tilted arrangement.

According to a fourth embodiment, the microfluidic device 100 is adapted for thermocycling, and there is disclosed a thermocycler (not shown) that incorporates the microfluidic device 100 of any of the above described embodiments, depending on the suitability for an intended application, as will be understood by persons skilled in the art.

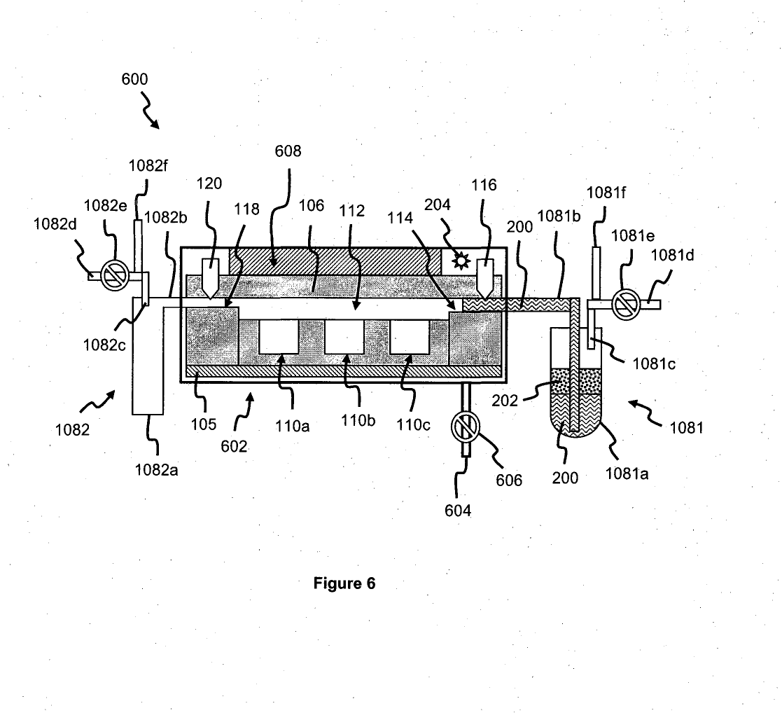

According to a fifth embodiment of a microfluidic device 600 as shown in FIG. 6, the assembled portion comprising the cover 106 and microtiter plate 102 (as described in the first embodiment) is held within an enclosure chamber 602, which is configured to support a vacuum environment. In particular, the enclosure chamber 602 has a sturdy arrangement and includes an inlet 604 to enable a third vacuum source (not shown) to be connected thereto for generating a vacuum in the enclosure chamber 602. The inlet 604 also includes an associated pressure regulator 606. Otherwise, the remaining arrangements of the microfluidic device 600 are similar to the first embodiment, and thus will not be repeated. The purpose of the enclosure chamber 602 is to enable a vacuum environment of a desired vacuum pressure to be generated and adjustable (using the associated pressure regulator 606) therewithin, whenever the space 112 (in the microfluidic device 100) is subjected to a differential pressure (in any of Steps 4A to 4D or Steps 5A to 5D). Importantly, the vacuum pressure generated within the enclosure chamber 602 at a specific instance is to be similar as a differential pressure formed within the space 112 at that instance, so that an external air pressure surrounding the cover 106 and base of the microtiter plate 102 are substantially equalised to the differential pressure to minimise warping of the cover 106 and base of the microtiter plate 102. Otherwise, warping of the cover 106 and base of the microtiter plate 102 can occur if a difference in air pressure exists between the external air pressure and that in the space 112. Further, a flat and substantially rigid top member 608 is also removably attached to the cover 106 of the microfluidic device 100 to provide another added measure for minimising warping of the cover 106. Indeed, the rigid top member 608 is of a similar form-factor and construction to the rigid base member 105 attached to the base of the microtiter plate 102. However, it is also to be appreciated that with inclusion of the rigid top member 608 and rigid base member 105 to minimise warping of the cover 106 and base, the internal of the enclosure chamber 602 can instead be configured at atmospheric pressure instead of vacuum pressure.

According to a sixth embodiment, which is a modification of the fifth embodiment, the first, second and third vacuum sources are replaced by a single common vacuum source. That is, the air port 1081d of the first vacuum generator 1081, the air port 1082d of the second vacuum generator 1082, and the inlet 604 of the enclosure chamber 602 are all coupled to the single common vacuum source. It will however be appreciated that desired air/vacuum pressures to be respectively formed at the air inlet 1081c of the first vacuum generator 1081, the air inlet 1082c of the second vacuum generator 1082, and in the enclosure chamber 602 can independently be tuned by adjusting the corresponding associated pressure regulators 1081e, 1082e, 606.

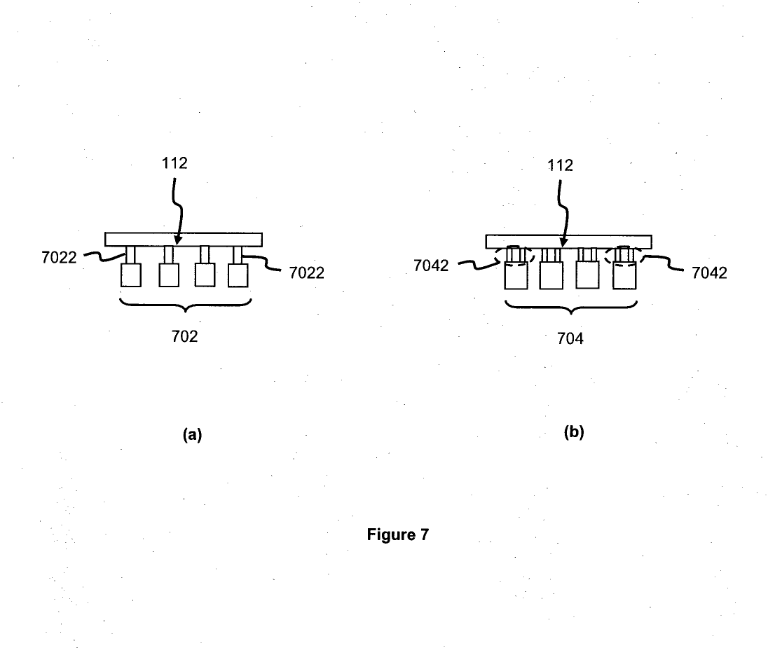

According to a seventh embodiment, which is similar to the first embodiment, but with a difference in configuration of the individual wells 110. Specifically, FIG. 7a shows a first possible configuration of wells 702, in which each well 702 is formed with a connecting channel 7022 that opens adjacently into the space 112 of the microfluidic device 100. That is, the well 702, connecting channel 7022 and space 112 are in fluid communication. On the other hand, FIG. 7b shows a second possible configuration of wells 704, in which each well 704 is instead formed with dual connecting channels 7042 that open adjacently into the space 112 of the microfluidic device 100. Thus, the well 704, dual connecting channels 7042 and space 112 are indeed in fluid communication. Notwithstanding FIGS. 7a and 7b, it will however also be apparent that other suitable configurations of wells are possible depending on application.

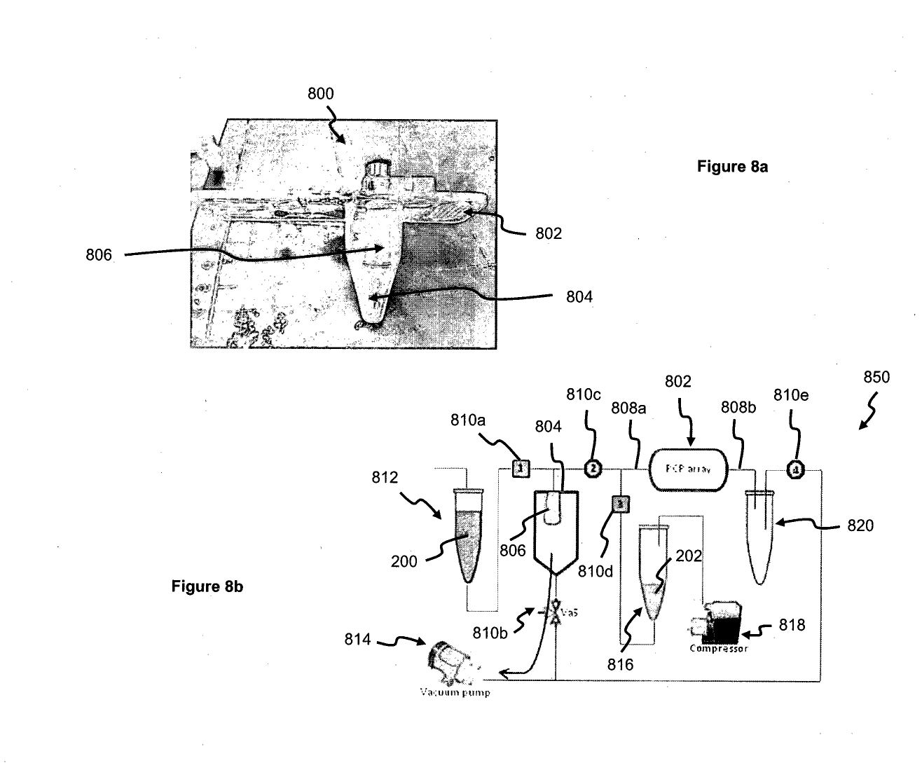

FIGS. 8a and 8b show an eighth embodiment of a microfluidic device 800. The microfluidic device 800, in this instance, comprises an array of wells 802, a vacuum chamber 804 and a reversibly deformable pouch 806 arranged within the vacuum chamber 804 in a substantially airtight configuration. Specifically, the pouch 806 is arranged to hold fluid sample 200, in which under influence of a higher ambient pressure, the pouch 806 is caused to compress thereby reducing the internal volume of the pouch 806 to squeeze fluid sample 200 out thereof.

The array of wells 802 is similar in arrangement and construction to the array of wells 110 of the first embodiment, and includes opening into the adjacent space 112 (similar to the first embodiment). Further, the array of wells 802 has an inlet channel 808a and outlet channel 808b to allow fluid sample 200 to be introduced thereinto. FIG. 8b then shows a schematic arrangement 850 of the microfluidic device 800 for use. Particularly, the pouch 806 is in fluid communication, though a first valve 810a, with a first chamber 812 holding the fluid sample 200. The vacuum chamber 804 is then coupled to a vacuum pump 814 (through a second valve 810b which includes a vacuum valve and a release valve), and the array of wells 802 is coupled at the inlet channel 808a to both the pouch 806 (through a third valve 810c) and to a second chamber 816 (through a fourth valve 810d) holding the sealant 202. The array of wells 802 is in fluid communication with the second chamber 816. The second chamber 816 is also coupled to a compressor 818. On the other hand, the array of wells 802 is also coupled at the outlet channel 808b to an empty third chamber 820, in which the third chamber 820 is also coupled to the vacuum pump 814 (through a fifth valve 810e). That is, the array of wells 802 is also in fluid communication with the third chamber 820.

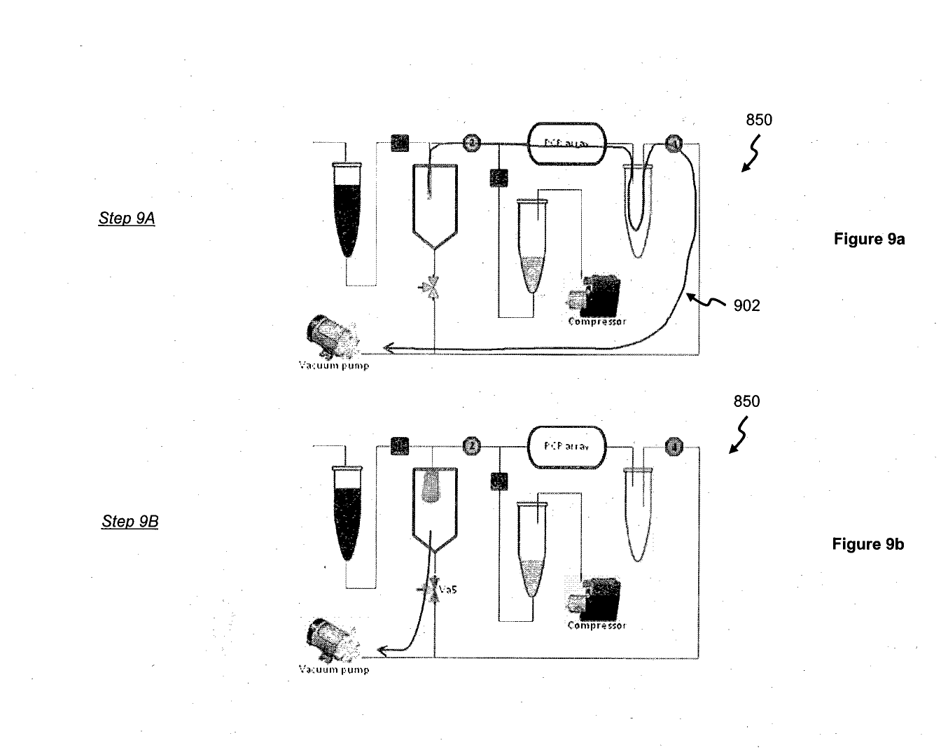

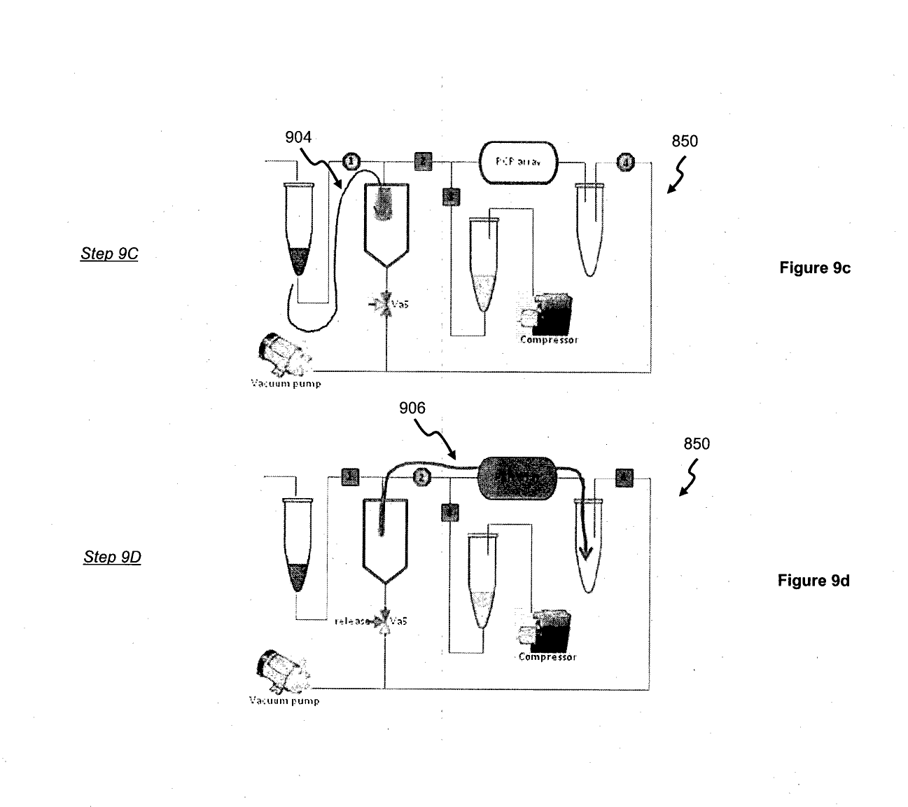

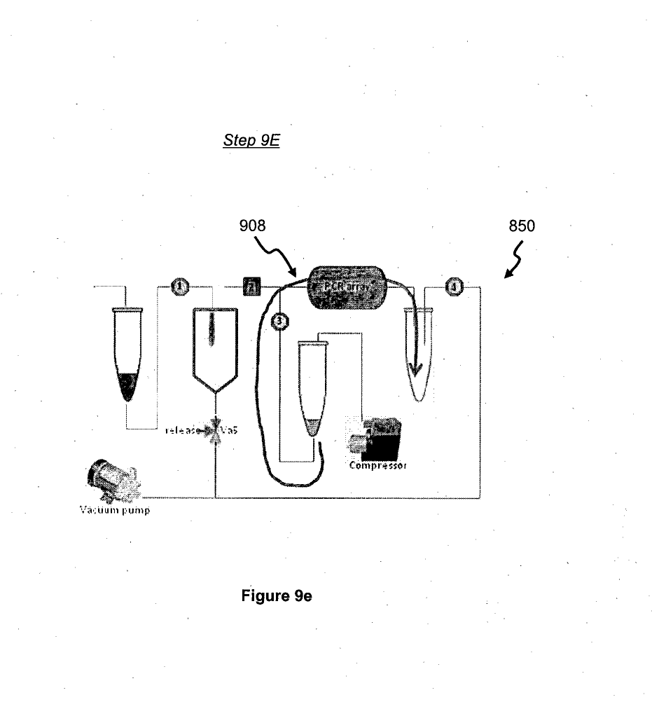

According to the eighth embodiment, FIGS. 9a to 9e collectively illustrate a method for using the microfluidic device 800. It is to be appreciated that before the method is commenced, all the valves 810a˜810e are initially closed. At Step 9A, the first valve 810a and fourth valve 810d are closed, whereas the release valve of the second valve 810b is opened to expose the vacuum chamber 804 to atmospheric pressure. Also, the third valve 810c and fifth valve 810e are opened, and the vacuum pump 814b is thereafter actuated to generate a vacuum, which consequently enables any air present in the pouch 806, inlet channel 808a, outlet channel 808b and array of wells 802 to be drawn into the third chamber 820 (i.e. as indicated by direction of arrow 902). It is highlighted that the pouch 806 is thus in a deflated shape at this instance.

At next Step 9B, the release valve of the second valve 810b closed, while the vacuum valve of the second valve 810b is then opened to the vacuum pump 814b in order for the same vacuum generated in Step 9A to be formed in the vacuum chamber 804 of the microfluidic device 800. The remaining valves 810a, 810c˜810e are kept in the same state as in Step 9A. This subsequently restores the pouch 806 to its original inflated shape due to equilibrium pressure being formed internal and external of the pouch 806. Progressing to Step 9C, the third valve 810c is then closed, and the first valve 810a is opened in order is for the fluid sample 200 held in the first chamber 812 to be drawn into and filling the pouch 806 (i.e. as indicated by direction of arrow 904).

At Step 9D, the first valve 810a is now closed, while the third valve 810c is opened to allow the fluid sample 200 held within the pouch 806 to move into the array of wells 802 (i.e. as indicated by direction of arrow 906) under influence of a differential vacuum, which is created by virtue of a pressure in the vacuum chamber 804 of the microfluidic device 800 is higher than a pressure in the third chamber 820. It is to be appreciated that the space 112 is filled with the fluid sample 200 during the process of filling the array of wells 802. To create the required differential vacuum, the vacuum valve of the second valve 810b is closed, while the release valve of the second valve 810b is opened to allow a small amount of air to enter the vacuum chamber 804 of the microfluidic device 800. Importantly, it is to be appreciated that a speed of flow of the fluid sample 200 into the array of wells 802 is controllable by adjusting an air flow rate entering the vacuum chamber 804 of the microfluidic device 800 through the release valve of the second valve 810b.

At Step 9E, the third valve 810c is now closed whereas the fourth valve 810d is opened, and the compressor 818 is actuated to generate a driving pressure. The driving pressure generated then pushes the sealant 202 held in the second chamber 816 into (the space 112 and) the array of wells 802 (i.e. as indicated by direction of arrow 908) to seal the wells 802. During this sealing process, the sealant 202 hence pushes the fluid sample 200 out of the space 112 into the third chamber 820. Further, any excess fluid sample 200 in the array of wells 802 are also pushed into the third chamber 820.

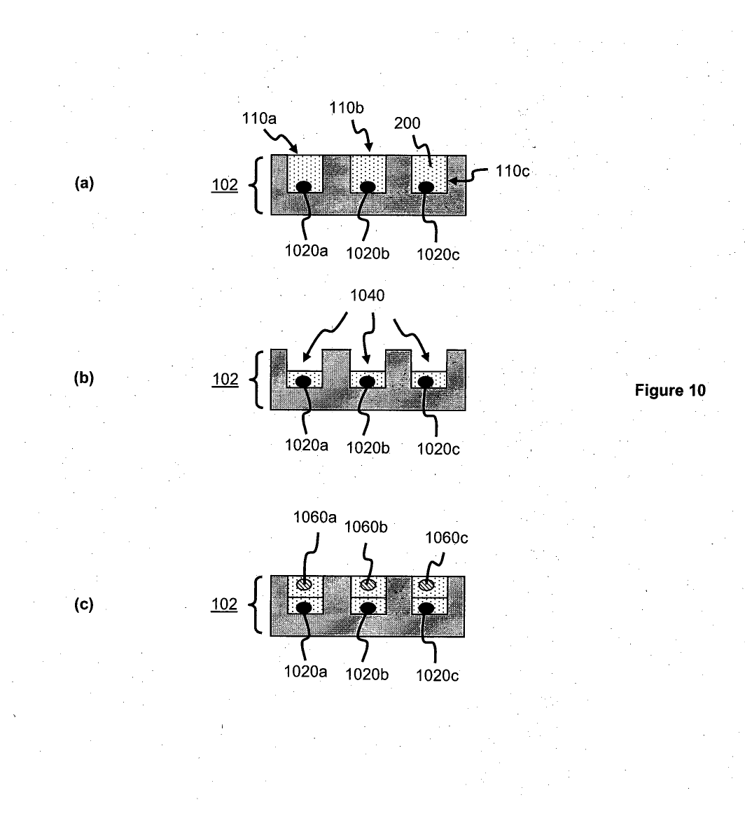

According to a ninth embodiment as shown in FIGS. 10a to 10c, the configuration of the microfluidic device 100 remains the same as in the first embodiment, but differing slightly in steps for loading biological/chemical materials into the wells 110a, 110b, 110c. Primers are used as an example of the biological/chemical materials for illustration purposes. Specifically, the biological materials may include cells. As depicted in FIG. 10a, the wells 110a, 110b, 110c are preloaded with a first set of primers 1020a, 1020b, 1020c and filled with the fluid sample 200, as similarly described in the first embodiment. Thereafter, a portion of the fluid sample 200 in each well 110a, 110b, 110c is evaporated to create a space 1040 (on top of the respective primers 1020a, 1020b, 1020c) for loading a second set of primers 1060a, 1060b, 1060c, as shown in FIG. 10b. Loading of the created spaces 1040 with the second set of primers 1060a, 1060b, 1060c and filling out the spaces 1040 with the fluid sample 200, as shown in FIG. 10c, are also carried out in a similar manner as in the first embodiment. The sealant 202 is then introduced to seal the wells 110a, 110b, 110c, like in afore embodiments. Subsequently, the first set of primers 1020a, 1020b, 1020c may then chemically/biologically interact with the second set of primers 1060a, 1060b, 1060c within the respective wells 110a, 110b, 110c. It will be appreciated that this embodiment relates to loading of multiple samples of the biological/chemical materials, in contrast to loading of a single sample of the biological/chemical materials as afore described under the first embodiment.

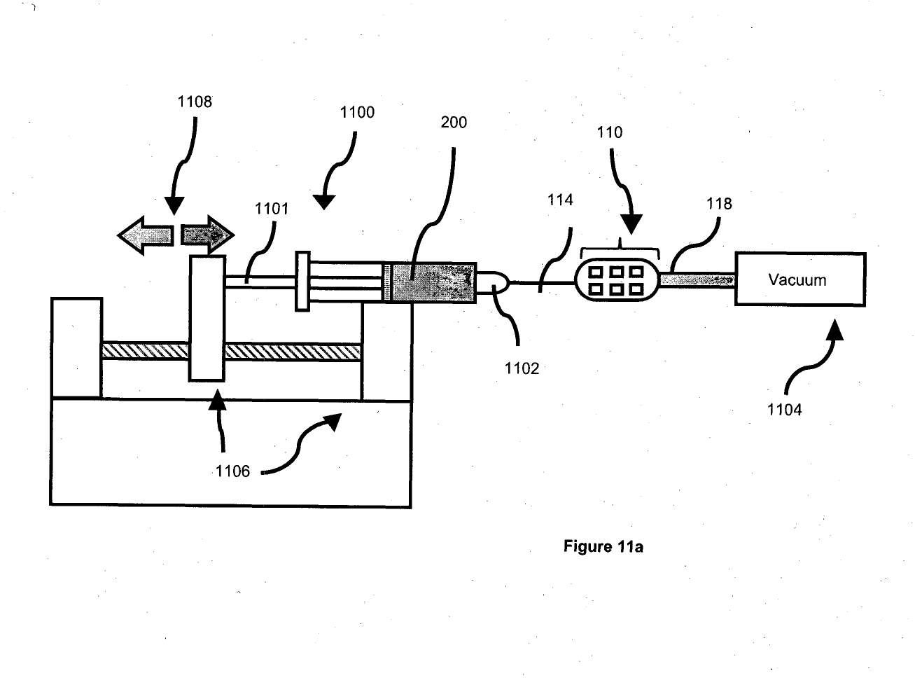

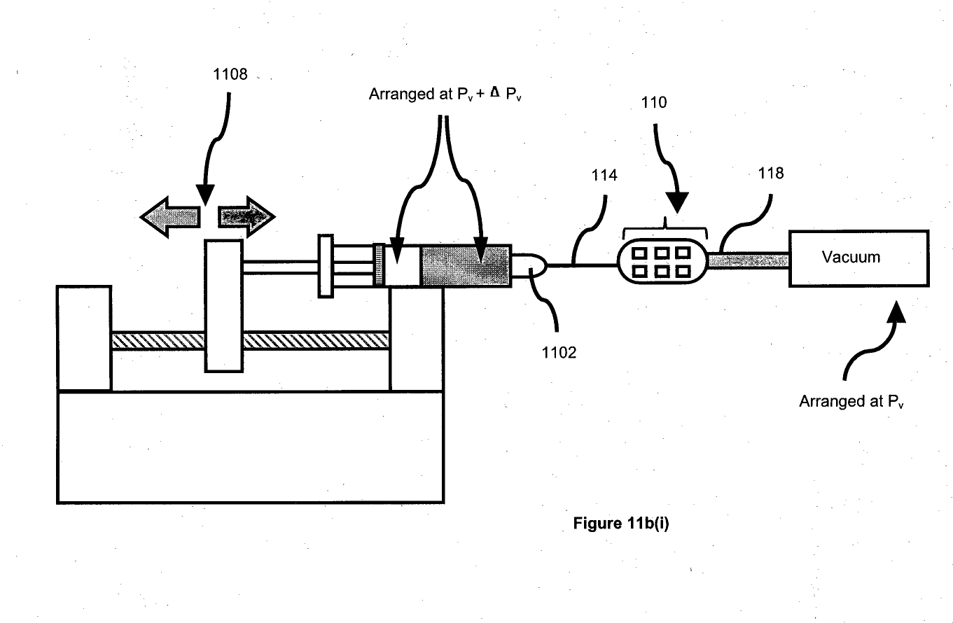

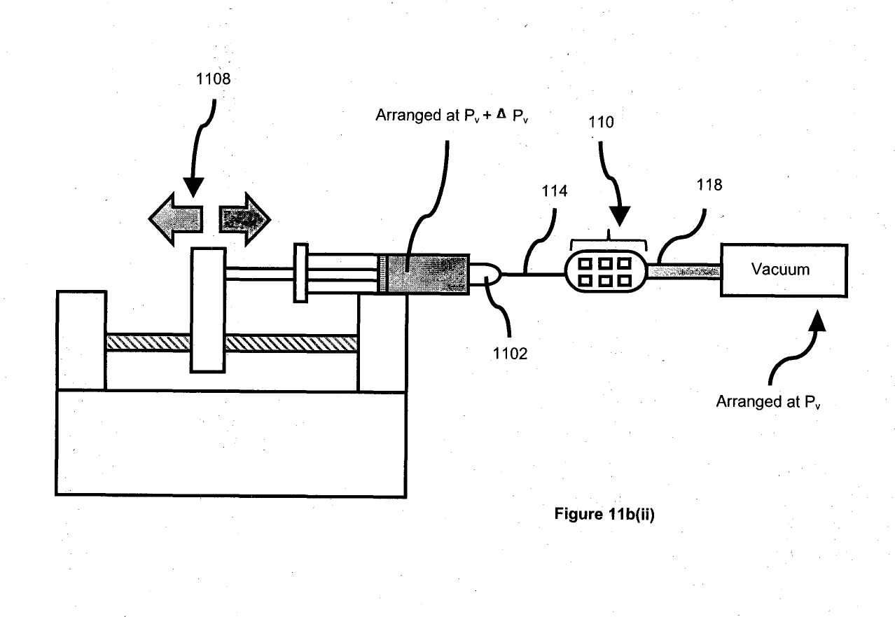

According to a tenth embodiment, FIG. 11a depicts a method for controlling a speed of introducing the fluid sample 200 into the array of wells 110 of the microfluidic device 100 of FIG. 1a. Specifically, a syringe 1100 (with a plunger 1101) is used to hold the fluid sample 200, and an open end 1102 of the syringe 1100 is adapted to be coupled to the inlet channel 114 connecting the array of wells 110. The open end 1102 of the syringe 1100 allows dispensing of the fluid sample 200. On the other hand, the outlet channel 118 connecting the array of wells 110 is coupled to a vacuum source 1104. In use, the vacuum source 1104 is actuated to generate a vacuum pressure of Pᵥ at the outlet channel 118, and a user (not shown) then restrains the plunger 1101 of the syringe 1100 using a syringe pump 1106 which is configured to controllably move the plunger 1101 forward at a desired speed, which gradually expels the fluid sample 200 out from the syringe 1100 into the array of wells 110. The pressure of the fluid sample 200 and an air space (if any), intermediate the fluid sample 200 and plunger 1101, is configured to be at Pᵥ+ΔPᵥ as depicted in FIG. 11b(i). Alternatively, only the pressure of the fluid sample 200 is configured to be Pᵥ+ΔPᵥ, if the plunger 1101 is immediately adjacent to the fluid sample 200 (i.e. no air space is present), as depicted in FIG. 11b(ii). It is appreciated that “forward” in the context of the preceding sentence means in a direction towards the open end 1102 of the syringe 1100. It will be apparent to skilled persons that if the plunger 1101 is not restrained using the syringe pump 1106, the plunger 1101 will instead move forward uncontrollably due to atmospheric pressure acting on the plunger 1101 and the presence of a vacuum at the outlet channel 118, which combines to generate a pressure differential that urges the plunger 1101 forward. Hence, by restraining the plunger 1101 using the syringe pump 1106 (i.e. forward/backward as indicated by arrows 1108), the user is able to controllably move the fluid sample 200 into the array of wells 110 at a desired speed.

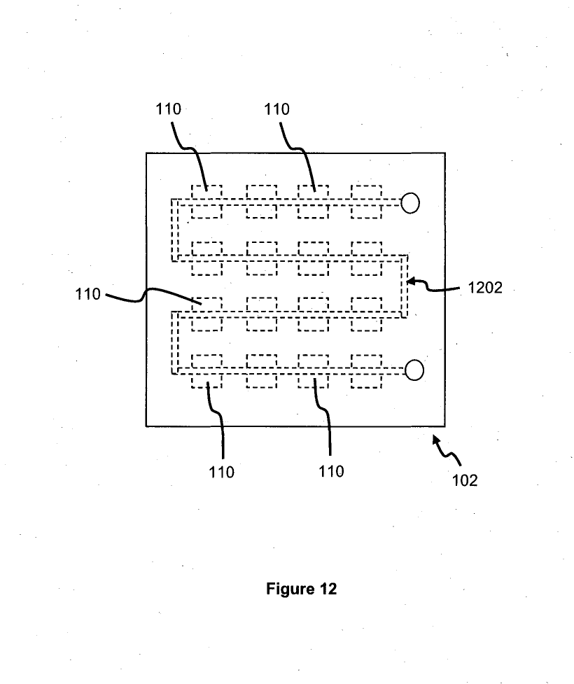

According to an eleventh embodiment, FIG. 12 shows the microtiter plate 102 of the microfluidic device 100 being configured with a longitudinal channel 1202 that is in fluid communication with each corresponding well 110 at respective sections along the length of the longitudinal channel 1202. That is, the longitudinal channel 1202 replaces the space 112, arranged adjacent to the wells 110, of the first embodiment. It will be appreciated that, in this instance, the longitudinal channel 1202 is also arranged adjacent to and above the wells 110. To form the longitudinal channel 1202 on a cover 106′ (not shown) that is fitted to the microtiter plate 102 (like as described in the first embodiment), the longitudinal channel 1202 is arranged and formed in an appropriate winding configuration that enables fluid connection to all the wells 110 of the microtiter plate 102. Other types of winding configuration or parallel channels are however also possible. It is highlighted that the arrangement of the longitudinal channel 1202, as described, helps to further reduce sample wastage from the wells, specifically during when Step 4D of the first embodiment or Step 5D of the second embodiment is carried out.

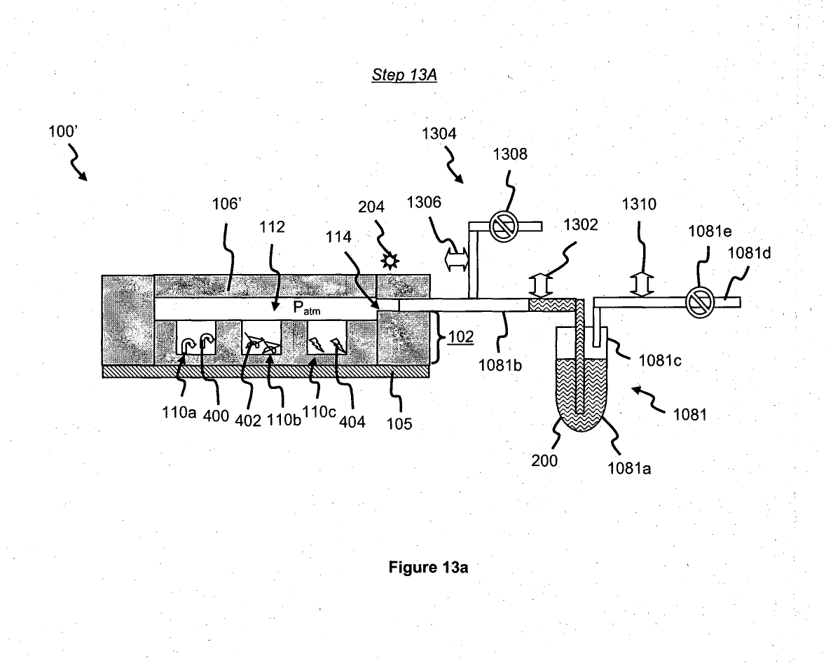

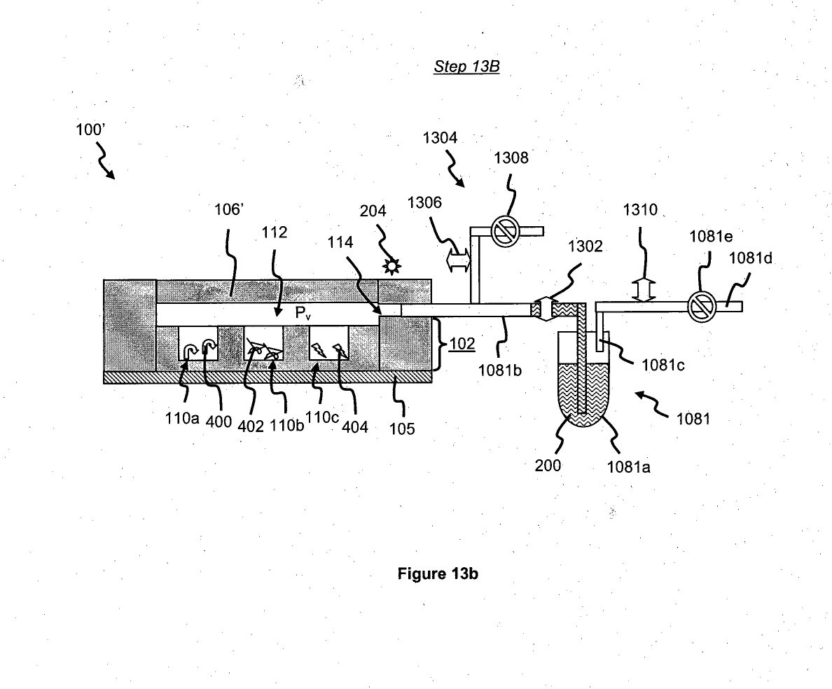

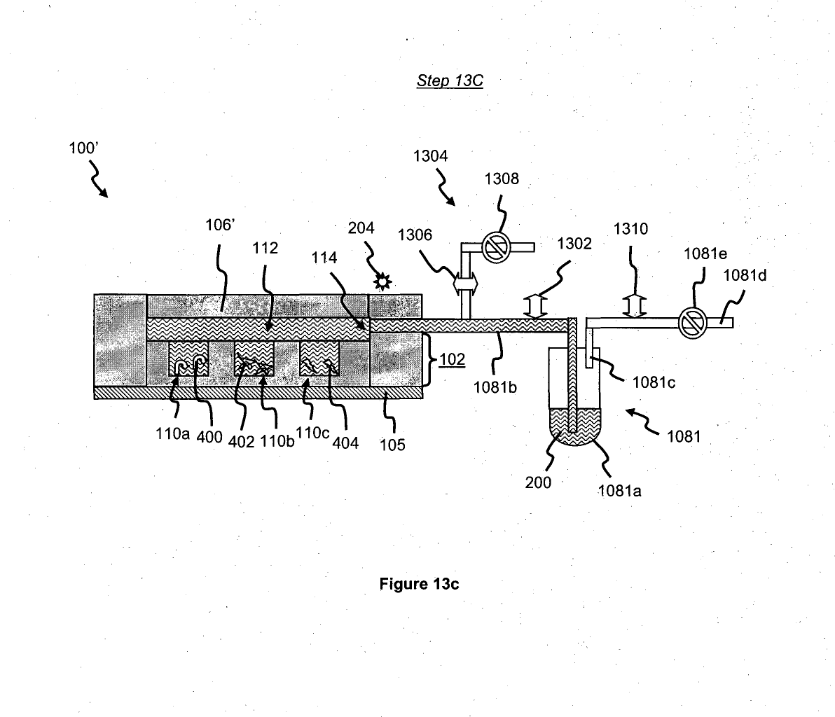

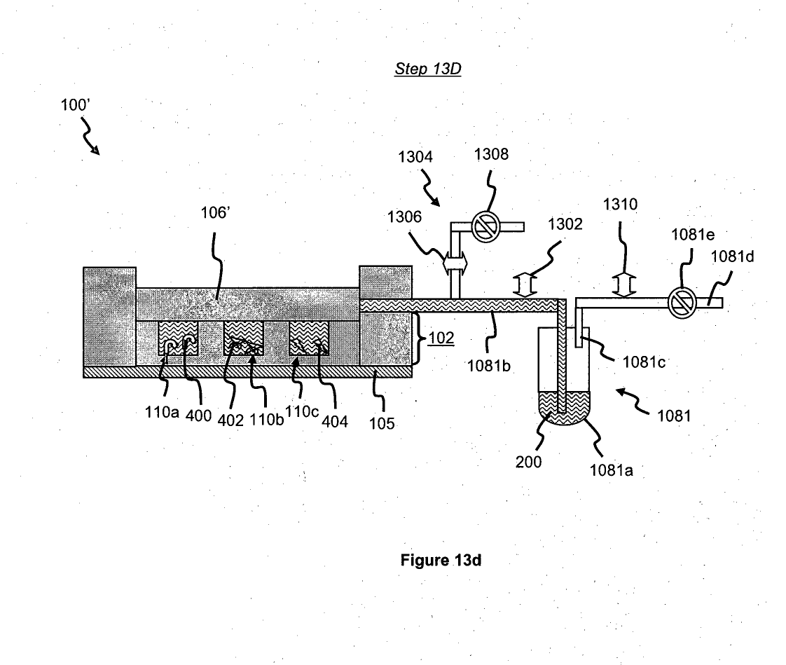

Referring to a twelfth embodiment, as shown in FIG. 13a, the space 112 is configured with only one single inlet, as opposed to two inlets (i.e. the inlet channel 114 and outlet channel 118), in which the single inlet is used to facilitate generation of the differential pressure, and introduction/withdrawal of the fluid sample 200/sealant 202 into/from the space 112, similar to the functionalities of the inlet channel 114 and outlet channel 118 as afore described. This also means that the vacuum generating device 108 now includes only a single vacuum generator coupled to the space 112 through the single inlet. In this embodiment, the inlet channel 114 is the aforementioned single inlet and the first vacuum generator 1081 is arranged to be the single vacuum generator (i.e. refer to FIGS. 13a to 13d). Accordingly, it will then be apparent that for the single vacuum generator, there is only a set of the following: a chamber, an inlet tubing, an air inlet, an air port, and a pressure regulator. This embodiment is described in greater detail in the next few paragraphs.

According to the twelfth embodiment, FIGS. 13a to 13d collectively illustrate another method, comprising Steps 13A to 13D, for introducing the fluid sample 200 into the space 112 to fill the wells 110a, 110b, 110c (which are respectively preloaded with the different types of primers 400, 402, 404) and thereafter sealing the filled wells 110a, 110b, 110c. It is highlighted that the microfluidic device 100′ in this embodiment is similar to the microfluidic device 100 in the first embodiment except that the air intake port 1081f of the first vacuum generator 1081 together with the second vacuum generator 1082 have been removed, and the outlet channel 118 that connects to the air port 1082d of the second vacuum generator 1082 is hermetically sealed. In addition, inlet tubing 1081b of the first vacuum generator 1081 is further configured with a first valve 1302 and an air inlet 1304. The air inlet 1304 is arranged with a second valve 1306 and an associated pressure regulator 1308, in which the second valve 1306 is positioned closer to the inlet tubing 1081b of the first vacuum generator 1081 than the pressure regulator 1308. Further, a third valve 1310 is also configured and positioned between the pressure regulator 1081e and air inlet 1081c of the first vacuum generator 1081. Also, a movable/deformable cover 106′ replaces the cover 106 of the first embodiment. Specifically, the movable/deformable cover 106′ can be movably lowered (e.g. using a plunger) adjacent to the wells 110a, 110b, 110c for sealing them, after being filled with the fluid sample 200. That is, the movable/deformable cover 106′ is adapted to be moved to reduce the size of the space 112.

In Step 13A, all the valves 1302, 1306, 1310 are initially opened, and the air inlet 1304 is opened to atmospheric pressure. As a result, the space 112 of the microfluidic device 100′ is exposed to atmospheric pressure. Compressed air is then applied to the air port 1081d of the first vacuum generator 1081 to push the fluid sample 200 into the inlet tubing 1081b thereof. Note that the fluid sample 200 is not yet introduced into the inlet channel 114 at this step. Next, in Step 13B, the first valve 1302 is closed, and a first pressure, Pᵥ, is applied to the air inlet 1304. This causes the space 112 to be also exposed to the first pressure of Pᵥ. It is highlighted that the first pressure is lower than atmospheric pressure and in this instance is a vacuum pressure.

At Step 13C, the second valve 1306 is closed and the first valve 1302 is now opened. A second pressure of Pᵥ+ΔPᵥ is then applied to the air port 1081d of the first vacuum generator 1081 (and also suitably adjusted using the pressure regulator 1081e). It is highlighted that the second pressure is lower than atmospheric pressure and in this instance is also a vacuum pressure. A differential pressure is then generated due to a difference in absolute pressure between the first pressure in the space 112 and second pressure at air port 1081d of the first vacuum generator 1081. This differential pressure then further urges the fluid sample 200 to move from the inlet tubing 1081b into the inlet channel 114 and space 112, until the fluid sample 200 completely fills the space 112 and wells 110a, 110b, 110c. Once that is attained, at final Step 13D, atmosphere pressure now replaces the second pressure for application to the air port 1081d of the first vacuum generator 1081 to stop urging the fluid sample 200 into the space 112, and the movable/deformable cover 106′ is then movably lowered to seal the wells 110a, 110b, 110c. It will be appreciated that as the movable/deformable cover 106′ is progressively lowered into the space 112, the fluid sample 200 residing in the space 112 is consequently squeezed out (into draining channels). Alternatively, the fluid sample 200 in the space 112 may be drained (e.g. into the chamber 1081a of the first vacuum generator 1081) before the movable/deformable cover 106′ is lowered to seal the wells 110a, 110b, 110c.

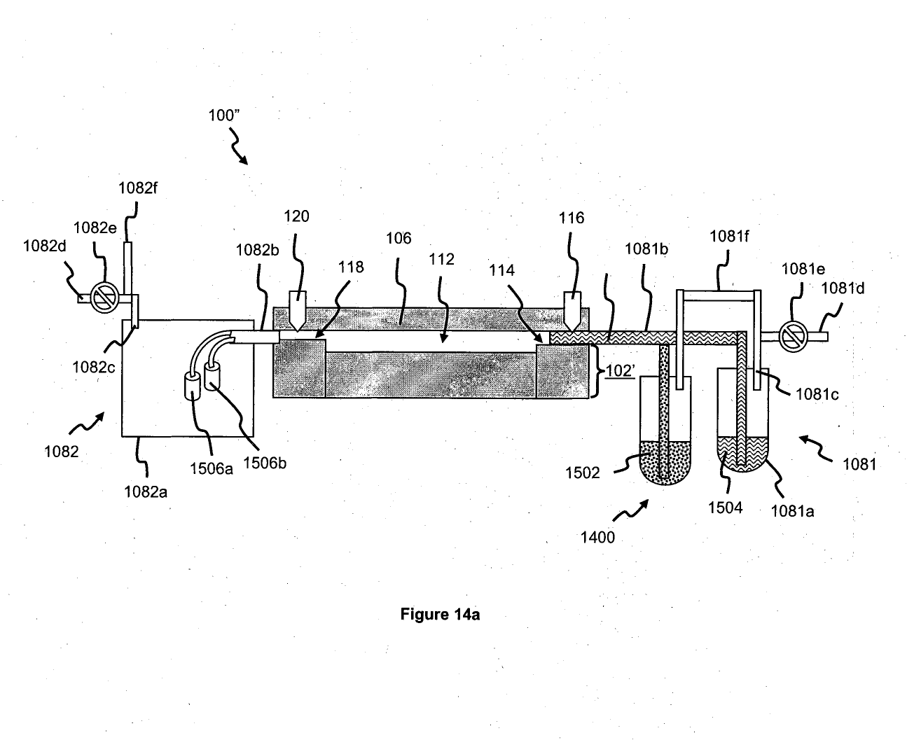

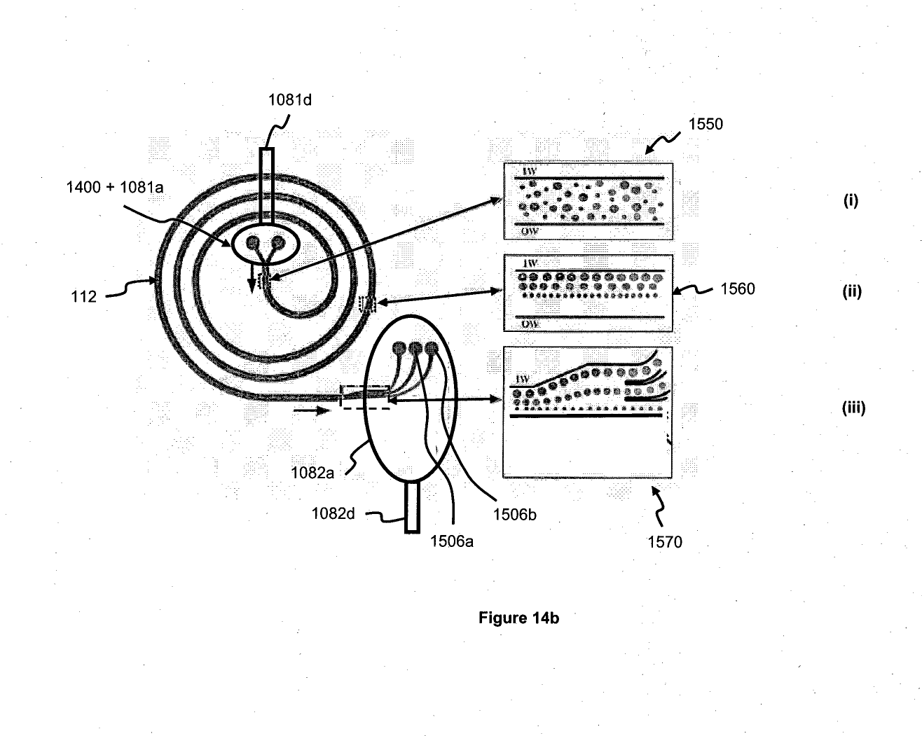

According to a thirteenth embodiment (refer to FIGS. 14a and 14b), the configuration of the microfluidic device 100″ remains the same as in the first embodiment, but with the following differences. Firstly, a microtiter plate 102′ does not have an array of wells and is also not attached to any rigid base member, but is otherwise similar to the microtiter plate 102 of the first embodiment. Secondly, a fluid sample 1502 (which contains different types of biological cells,) is held in an external sealant dispenser 1400 while the air intake port 1081f of the first vacuum generator 1081 has been extended to the sealant dispenser 1400. That is, the air intake port 1081f has been extended and reconfigured for coupling the sealant dispenser 1400 to the air port 1081d of the first vacuum generator 1081: In addition, the chamber 1081a of the first vacuum generator 1081 now optionally holds a buffer fluid 1504 which helps to assist in the separation of biological cells of different sizes.

A motivation for this embodiment is that it is desirable to remove air bubbles in the regions of the inlet channel 114, outlet channel 118, and the flow path before liquid flow or start of a process for loading the fluid sample 1502. It is to be appreciated that a method of loading the fluid sample 1502 without air bubbles using the microfluidic device 100″ of the current embodiment is the same as afore described in the first embodiment, and thus for sake of brevity, will not be repeated. It is however also to be understood that during the operation of loading the fluid sample 1502, the first pressure of Pᵥ is applied to both the sealant dispenser 1400 and the chamber 1081a of the first vacuum generator 1081.

Further, it is also to be appreciated that in other embodiments that may be envisaged, respective syringe pumps can be used in place of the external sealant dispenser 1400 and the chamber 1081a of the first vacuum generator 1081. Further, a plurality of tube holders (i.e. refer to FIG. 15b) are coupled to the outlet tubing 1082b of the second vacuum generator 1082, wherein only two of those tube holders 1506a, 1506b are depicted in FIG. 15a, for sake of brevity. It is also to be specifically highlighted that for this embodiment, the microfluidic device 100″ is configured with at least two flow outlets branching from the outlet tubing 1082b of the second vacuum generator 1082 for biological cells collection using at least two of the tube holders 1506a, 1506b. Specifically, the at least two flow outlets and the associated plurality of tube holders are individually configured for collecting different types of biological cells of different sizes, and the plurality of tube holders are housed within the chamber 1082a of the second vacuum generator 1082. In addition, the space 112 is now arranged in a spiral-like configuration (although straight or other suitable configurations can also be used if necessary), as depicted in FIG. 15b. Yet further, the space 112 may also be adapted for separation of particles, such as different types of biological cells, for example based on their respective sizes, when the fluid sample 1502 is subsequently introduced to flow along within the space 112. In this embodiment, the space 112 is specifically configured as a conduit.

It will be appreciated that a method of loading the fluid sample 1502 and the buffer fluid 1504 without air bubbles using the microfluidic device 100″ of the current embodiment is the same as afore described in the first embodiment (refer to FIG. 1a), and thus for sake of brevity, will not be repeated. Importantly, the method using the current setup of the microfluidic device 100″ of this present embodiment enables the biological cells in the fluid sample 1502 to be substantially sorted in respective sections along the length of the space 112, based on their individual weight and size. Specifically, the different biological cells are initially mixed together within the fluid sample 1502 as seen from FIG. 15b(i), when the fluid sample 1502 is initially introduced into the space 112. Subsequently as the fluid sample 1502 flows along the length of the space 112 towards the plurality of tube holders, the different biological cells are then automatically sorted at different sections along the length of the space 112, due to the respective sizes and weight of different biological cells, which consequently affects their speed of flow along the space 112. This is depicted in FIG. 15b(ii). In particular, the larger biological cells are more likely to be deposited closer to the inner face of the space 112, while the smaller biological cells are then more likely to be deposited closer to the outer face of the space 112. In this context, the inner face of the space 112 is defined to be always closer to the center of the spiral-like arrangement of the space 112 than the outer face of the space 112. That is, a radius defined from the center of the spiral-like arrangement of the space 112 to the inner face of the space 112 is always shorter than an equivalent one defined to the outer face of the space 112. Thereafter, the different sorted biological cells in the fluid sample 1502 can then be collected in the respective tube holders as shown in FIG. 15b(iii).

It is also to be appreciated that the microfluidic device 100″ of the thirteenth embodiment can also be applied for usage to the particle separation channels as shown in FIGS. 1 to 6 of a journal paper titled: “Label-free cell separation and sorting in microfluidic systems”, published in Anal Bioanal Chem (2010) 397:3249-3267, by Daniel R. Gossett & Westbrook M. Weaver & Albert J. Mach & Soojung Claire Hur & Henry Tat Kwong Tse & Wonhee Lee & Hamed Amini & Dino Di Carlo.

In summary, the microfluidic device 100 and the corresponding methods (of the various embodiments as described) beneficially enable a speed of flow of the fluid sample 200/sealant 202 when being introduced into the space 112 to be controllable using differential pressure, and thus advantageously allows biological/chemical materials preloaded into the array of wells 110 to be retained therein during when the fluid sample 200/sealant 202 is introduced without risk of unintentionally being flushed out from the associated wells 110 that may result in undesirable cross-contamination of neighbouring wells 110. In particular, the speed of flow of the fluid sample 200/sealant 202 is controllable to be of a sufficiently slow speed to enable the aforementioned advantages to be achieved. Additionally, with reference to Steps 4C and 4D of FIGS. 4c and 4d, the slow speed of flow of the fluid sample 200 also enables the sealant 202 to substantially seamlessly adhere to the fluid sample 200 during introduction into the space 112 without the sealant 202 disintegrating into blobs. Yet further, the slow speed of flow of the sealant 202 advantageously prevents the content of filled wells 110 from being pulled out by the high shear stress forces generated at the fluid interface between the sealant 202 and the fluid sample 200 in the wells 110 (exposed at the openings thereof), that would otherwise drag out the fluid sample 200 (together with the preloaded biological/chemical materials) from the wells 110. On the other hand, the differential pressure as generated is sufficiently high which will also mitigate the problem of air pockets being trapped within the array of wells 110.

Also, it will be appreciated that the microfluidic device 100 and the corresponding methods enable control of the absolute vacuum pressure in the space 112 and connected wells independently from the flow speed of the fluid sample 200/sealant 202 through the space 112. Importantly, the flow speed of the fluid sample 200/sealant 202 is determined by the resulting differential between the first and second absolute pressures; that is, the vacuum pressure, Pᵥ, can be set in the space 112/wells 110a, 110b, 110c at a desired pressure level, whilst the flow speed of the fluid sample 200/sealant 202 is set independently to be at a desired speed level by varying the value of ΔPᵥ.

It is also to be appreciated that present invention can reduce wastage as the amount of fluid flowing through may be precisely controlled. Comparatively, existing vacuum-driven well loading devices with a “flow-through” channel and headspace, where a portion of the sample is sucked out of the headspace by the vacuum generated during device operation, causes sample wastage.

It is also to be appreciated that the chamber 1081a of the first vacuum generator 1081 which allows the sealant 202 to be held above the fluid sample 200 helps to eliminate any possible presence of air column between the fluid sample 200 and the sealant 202 (i.e. an air-sealant interface). Specifically, absence of the air-sealant interface beneficially prevents formation of any air pockets (in the space 112 or the array of wells 110) when the sealant 202 is subsequently introduced into the space 112. Another advantages of the microfluidic device 100 include being reliable for repeated usage, low cost and simple to manufacture using existing manufacturing techniques.

It is further appreciated that for the embodiments, where no wells 110 are configured in the microtiter plate 102, an advantage that may arise from this arrangement is that it will beneficially help to prevent the introduction and formation of air bubbles within the space 112 during loading of the fluid sample 200. Air entrapment at the inlet may also be avoided.

It is further highlighted that in the microfluidic device 100, the array of wells 110 are configured with their openings directly facing and connecting with the space 112 (i.e. the open wells arrangement). This open wells arrangement advantageously enables a higher well density for the microfluidic device 100 at lower costs, as well as providing improved reliability during manufacturing. Moreover, the open wells arrangement also provides improved performance reliability of the microfluidic device 100, since air pockets trapped within any of the wells 110 can more easily be released into the space 112 during device operation. Suitable applications for the microfluidic device 100 include PCR array, qPCR, digital PCR, single cell isolation/analysis or the like.

The described embodiments should not however be construed as limitative. For example, it will be understood that the array of wells 110 need not necessarily be preloaded with any biological/chemical materials prior to filling the wells 110 with the fluid sample 200. In such an instance, the fluid sample 200 to be subsequently introduced may then contain biological/chemical materials (in dried, partially dried, or liquid forms) including PCR primers (e.g. Oligonucleotides, short fragments of genes etc.), cells, viruses, antibodies, proteins, enzymes, molecules, peptides, polynucleotides, reaction constituents (e.g. double emulsion droplets), nucleic acid molecules (e.g. DNA, RNA, mRNA, microRNA, cDNA etc), bacteria, protozoa, pathogens, fluorescent chemicals/molecules, crystals etc.

Also, the space 112 above the array of wells 110 may alternatively be defined by walls (not shown) extending substantially vertically upwards from the base of the microtiter plate 102, as opposed to being defined by the cover 106 acting as a cover fitting over the base of the microtiter plate 102. Moreover, the microfluidic device 100 may also have a deformable/movable cover plate (e.g. made of rubber), in place of the cover 106, arranged to be pressed down (e.g. using a plunger) on the microtiter plate 102 for sealing the array of wells 110, thereby compressing and sealing the fluid sample 200 in the array of wells 110. In addition, the microtiter plate 102 may also optionally be equipped with an ID chip or a barcode for identification purposes, as will be apparent. Yet further, the microtiter plate 102 may also be configured with at least a single well, as opposed to the array of wells 110. Yet additionally, each well 110, instead of having a cuboid shape described in the first embodiment, may be formed with any suitable shape (e.g. cylindrically-shaped) based on different intended applications. Furthermore, the fluid flow sensor 204 may also be optional. Also, in certain embodiments, the sealant 202 used need not necessarily be less dense than the fluid sample 200. That is, the sealant 202 may be denser than the fluid sample 200 since the presence of surface tension due to the sufficiently small dimensions of the wells 110 would in fact prevent the denser sealant from sinking into the wells 110 to push out the fluid sample 200.

Yet in another variation, the microfluidic device 100 may further comprise a body container with a door (not shown), in which the body container is adapted to internally hold a plurality of the microtiter plates 102 at respective horizontal levels along the height of the body container. Specifically, each microtiter plate 102 is removably attached at a respective horizontal level within the body container. Also, the body container is formed and configured to support differential pressures environment therewithin, similar to the combination of the cover 106 when securely attached to the base of the microtiter plate 102 as described in the first embodiment. Moreover, the body container also similarly includes the necessary constructs (e.g. the inlet channel 114 and outlet channel 118) to support generation of a differential pressures therewithin using the vacuum generating device 108. In use, the body container is employed in a manner to collectively load the array of wells 110 of all microtiter plates 102 (held in the body container) with the fluid sample 200, and thereafter, the microtiter plates 102 may then be removed from the body container for further processing. The advantage of using the body container is thus enabling multiple microtiter plates 102 to be loaded with the fluid sample 200 in one single step, allowing for greater convenience and ease of operation.

It will also be appreciated that microfluidic device 100 may be integrated with upstream sample preparation and/or downstream analysis devices. For example, the microfluidic device 100 may be adapted for thermocycling in a thermocycler (as described in the fourth embodiment). Alternatively, only the microtiter plate 102, with the base of the microtiter plate 102 having the array of wells 110, may be removed and placed in the thermocycler, in which the microtiter plate 102 is beneficially optimised for efficient heat transfer through the base of the microtiter plate 102 to facilitate performance of nucleic acid amplification techniques (e.g. PCR).

Also, the rigid base member 105 may not be required for attachment to the base of the microtiter plate 102, if the mentioned base is formed of an appropriate material that is substantially rigid on its own to counter warping of the base, when an air pressure within the space 112 is dower than atmospheric pressure. In addition, the rigid base member 105 may also optionally be formed of other suitable materials such as glass or the like, and not necessarily aluminium.

Further optionally, rather than coupling the first and second vacuum generators 1081, 1082 to the single common vacuum source 104, separate first and second vacuum sources may alternatively be coupled to the first and second vacuum generators 1081, 1082 respectively. It will however be appreciated that as in the first embodiment, generation of a differential pressure within the space 112 and the wells 110a, 110b, 110c is still effected and controlled using the respective pressure regulators 1081e, 1082e of the first and second vacuum generators 1081, 1082.

Yet optionally, the first vacuum source is configured as a fixed vacuum source that outputs only a predetermined pressure level and is thus non-adjustable, whereas the second vacuum source remains of the same configuration as in the first embodiment. The preceding statement is conversely also true if the first vacuum source is instead maintained in the same configuration as in the first embodiment, whereas the second vacuum source is now arranged as a fixed vacuum source.

Alternatively, with Step 4C, the outlet control valve 120 may alternatively be maintained in the prior open position according to Step 4B, since the fluid sample 200 introduced into the space 112 will not flow (or rush) out easily from the outlet channel 118 into the chamber 1082a of the second vacuum generator 1082, as the outlet channel 118 is formed relatively narrower than the inlet channel 114 (as described in the first embodiment) to prevent easy inherent outflow of the fluid sample 200 from the space 112, in the absence of application of an urging force to push the fluid sample 200 out.

In using Step 4D of the first embodiment as an illustration, the air pressures of P₁ and P₂ need not be configured at the respective first and second vacuum levels; instead the air pressures of P₁ and P₂ may alternatively be configured to be at first and second compressed air pressures respectively. In particular, the air pressure of P₁ at the first compressed air pressure is higher than the air pressure of P₂ at the second compressed air pressure in order to drive the sealant 202 into the space 112 when the inlet and outlet control valves 116, 120 are switched to the open positions.

With reference to the second embodiment, the sealant 202 may also be introduced into the space 112 through the air inlet 1081c of the first vacuum generator 1081 or the air inlet 1082c of the second vacuum generator 1082, instead of via the additional channel 500 connected to the inlet tubing 1081b of the first vacuum generator 1081. Yet optionally, the microfluidic device 100 may also be further equipped with another channel (not shown), being similar to the additional channel 500 of the second embodiment, that is connected to the outlet tubing 1082b of the second vacuum generator 1082, and the sealant 202 may then accordingly be introduced through this another channel into the space 112. In this instance, the sealant 202 is also held in the external sealant dispenser as will be understood.

With regard to method Step 5D of the second embodiment, it is also to be appreciated that instead of withdrawing the fluid sample 200 from the space 112 before introducing the sealant 202 thereinto, the fluid sample 200 may alternatively be moved into the chamber 1081a of the first vacuum generator 1081 or the chamber 1082a of the second vacuum generator 1082 during as the sealant 202 is being introduced into the space 112. Indeed, whilst the sealant 202 is being introduced into the space 112, the sealant 202 pushes the fluid sample 200 out into the chamber 1081a of the first vacuum generator 1081 or the chamber 1082a of the second vacuum generator 1082, depending where the sealant 202 is being introduced. Yet further, in Step 5C of the second/third embodiment, the outlet control valve 120 may alternatively continue to be maintained in the open position as per Step 5B, as the fluid sample 200 is introduced into the space 112.

Alternatively, a specially adapted device (e.g. a robotic device) may be used to restrain and control the forward movement of the plunger 1101 (as described in the tenth embodiment of FIG. 11a). In this case, the restraining of the plunger 1101 may be automated. Optionally, the forward movement of the plunger 1101 may also be controllable manually using the user's hands.

With regard to the first embodiment, an optional step may be included after performance of Step 4C but prior to Step 4D. The optional step relates to further applying a pressure higher than Pᵥ+ΔPᵥ to the air inlet 1081c of the first vacuum generator 1081 in order to push the fluid sample 200 already in the wells 110a, 110b, 110c, for the benefit of overcoming any surface tension in the wells 110a, 110b, 110c, which consequently ensures that the entire space of the wells 110a, 110b, 110c is completely filled with the fluid sample 200. It is to be appreciated that references to the fluid sample 200 in the preceding sentence also include references to the sealant 202 under the appropriate context.

Further, in some embodiments that may be envisaged, Step 4D of the first embodiment may be optional since many cell-based assays do not require sealing of wells. In those instances, the space 112 may be empty or otherwise filled with aqueous buffer that may contain molecules or nutrients.

It is also to be appreciated that the fluid sample 20 includes constituents that enable biological or chemical assay (e.g. nucleic acid amplifications, cells assay, PCR etc) to occur with any materials pre-disposed in the wells 110. It is yet further to be appreciated that each of the plurality of wells 110 may optionally hold specific preloaded material different from those held in another well 110 for facilitating nucleic acid amplifications, such as polymerase chain reaction and other primer extensions, and/or assays related to cells and proteins. The material may include cells, proteins and oligonucleotides.

It is further to be appreciated that if the vacuum pressure generated by the single common vacuum source 104 is relatively stable, then it may be possible to do away with either the pressure regulator 1081e of the first vacuum generator 1081 or the pressure regulator 1082e of the second vacuum generator 1082 since one of the vacuum generators 1081, 1082 can instead inherit the vacuum pressure of the single common vacuum source 104, with no adjustment then required for the mentioned vacuum generator 1081, 1082. Thus accordingly, no pressure regulator 1081e, 1082e is needed for that associated vacuum generator 1081, 1082.

While the invention has been illustrated and described in detail in the drawings and foregoing description, such illustration and description are to be considered illustrative or exemplary, and not restrictive; the invention is not limited to the disclosed embodiments. Other variations to the disclosed embodiments can be understood and effected by those skilled in the art in practising the claimed invention.

1. A microfluidic device comprising:
a member with a base having at least one well, the at least one well in fluid communication with an adjacent space, said space being in fluid communication with at least one channel; and
a vacuum generating device coupled to the at least one channel, wherein the vacuum generating device is configured to generate first and second absolute pressures that are each lower than atmospheric pressure at a first and a second region of the microfluidic device respectively, wherein the first absolute pressure is higher than the second absolute pressure, thus generating a differential pressure between the first and second regions of the microfluidic device to control a speed of a fluid flowing through the space of the device, for filling the at least one well progressively and/or facilitating retention of any material disposed in the at least one well.

2. The device of claim 1, wherein the vacuum generating device comprises at least two vacuum generators in cooperative arrangement to generate the differential pressure.

3. The device of claim 2, wherein the at least one channel includes at least first and second channels, wherein a first vacuum generator is coupled to the at least first channel being an inlet channel for the fluid to flow into the space adjacent the at least one well and a second vacuum generator is coupled to the at least second channel being an outlet channel for the fluid to flow out of the space adjacent the at least one well.

4. The device of claim 3, wherein the first vacuum generator is configured to generate the first absolute pressure in the vicinity of the inlet channel and the second vacuum generator is configured to generate the second absolute pressure in the vicinity of the outlet channel to control the speed of fluid flow into the space adjacent the at least one well.

5. The device of claim 3, wherein at least one of the two vacuum generators includes a pressure regulator configured to enable independent adjustment of pressure in the vicinity of the inlet channel or the outlet channel.

6-7. (canceled)

8. The device of claim 1, further comprising at least one control valve disposed adjacent to the at least one channel to control fluid access into the space.

9. The device of claim 3, comprising at least a first control valve, disposed adjacent to the inlet channel, adjustable to allow fluid access to the space and at least a second control valve, disposed adjacent to the outlet channel, adjustable to allow the fluid to flow out of the space.

10. The device of claim 3, wherein the differential pressure causes the fluid to flow through the space from the inlet channel to the outlet channel.

11. The device of claim 1, wherein the at least one well is in fluid communication with the space by being connected to the space via at least one channel.

12. The device of claim 1, further comprising a cover for the member and top and bottom rigid members removably attached to respectively the cover and member of the device to prevent warping under influence of the differential pressure during operation.

13-19. (canceled)

20. A method of controlling fluid flow within a microfluidic device comprising a member with a base having at least one well, the at least one well in fluid communication with an adjacent space, said space being in fluid communication with at least one channel; and a vacuum generating device coupled to the at least one channel, the method comprises:
generating first and second absolute pressures that are each lower than atmospheric pressure at a first and a second region of the microfluidic device respectively, wherein the first absolute pressure is higher relative to the second absolute pressure thus generating a differential pressure between the first and second regions of the microfluidic device to control a speed of a fluid flowing through the space of the device, for filling the at least one well progressively and/or facilitating retention of any materials that are disposed in the at least one well.

21. The method of claim 20, comprising using the vacuum generating device which comprises at least two vacuum generators in cooperative arrangement to generate the differential pressure.

22. The method of claim 21, comprising using at least a first vacuum generator coupled to at least a first channel being an inlet channel for the fluid to flow into the space adjacent to the at least one well to generate the first absolute pressure in the vicinity of the inlet channel and at least a second vacuum generator coupled to at least a second channel being an outlet channel for the fluid to flow out of the space adjacent the at least one well to generate the second different absolute pressure in the vicinity of the outlet channel to control the speed of fluid flow into the space adjacent to the at least one well, wherein the at least one channel includes the at least first and second channels.

23. The method of claim 22, wherein at least one of the vacuum generators includes a pressure regulator to independently adjust the first absolute pressure or second absolute pressure.

24. The method of claim 20, further comprising controlling fluid access into the space via at least one control valve disposed adjacent to the at least one channel.

25. The method of claim 22, comprising allowing fluid access into the space by using at least a first control valve disposed adjacent to the inlet channel and allowing the fluid to flow out of the space by using at least a second control valve disposed adjacent to the outlet channel.

26. The method of claim 20, comprising:
introducing a sealant to substantially replace the fluid in the space subsequent to substantially filling the at least one well with the fluid; and
filling the space with the sealant to seal the at least one well substantially filled with the fluid,
wherein the sealant is introduced using the generated differential pressure or compressed air configured to be of a substantially high pressure for further pushing the fluid filled in the at least one well into any unoccupied space in the at least one well as the sealant fills the space.

27. The method of claim 20, comprising:
substantially removing the fluid from the space subsequent to substantially filling the at least one well with the fluid; and
introducing a sealant into the space to seal the at least one well substantially filled with the fluid,
wherein the sealant is introduced using the generated differential pressure or compressed air configured to be of a substantially high pressure for further pushing the fluid filled in the at least one well into any unoccupied space in the at least one well as the sealant fills the space.

28. The method of claim 26, comprising introducing the sealant from a common container containing both the fluid and the sealant or, introducing the fluid from a first container and introducing the sealant from a separate second container containing the sealant only.

29-30. (canceled)

31. A method of controlling fluid flow within a microfluidic device comprising a member with a base having at least one well, the at least one well in fluid communication with an adjacent space, said space being in fluid communication with inlet and outlet channels; a fluid dispensing device coupled to the inlet channel; and a vacuum generating device coupled to the outlet channel, the method comprises:
using the vacuum generating device to generate an absolute pressure lower than atmospheric pressure in the vicinity of the outlet channel; and
operating the fluid dispensing device to provide an absolute pressure in the vicinity of the inlet channel which is lower than atmospheric pressure but higher than the absolute pressure in the vicinity of the outlet channel, thus generating a differential pressure to control a speed of flow of the fluid into the space for filling the at least one well progressively and/or facilitating retention of any material disposed in the at least one well.

32-41. (canceled)
